(19) United States
(12) Patent Application Publication
(10) Pub. No.: US 2025/0263396 A1
(43) Pub. Date: Aug. 21, 2025

(54) CRYSTALLINE FORMS OF 3-(5-(2-HYDROXY-2- METHYLPROPOXY)-6-METHYPYRAZIN-2-YL)-1H-INDOLE-7-CARBONITRILE

(71) Applicant: NIDO BIOSCIENCES, INC., WATERTOWN, MA (US)

(72) Inventors: Bakary-Barry TOURE, Weston, MA (US); Anuj K. Kumar, Belmont, MA (US); Vladimir Volchenok, Waltham, MA (US); Magnus Ronn, Brownsville, VT (US)

(21) Appl. No.: 19/036,831

(22) Filed: Jan. 24, 2025

Related U.S. Application Data
(60) Provisional application No. 63/625,470, filed on Jan. 26, 2024.

(51) Int. Cl. C07D 403/04; A61K 31/497

(52) U.S. Cl. CPC C07D 403/04; A61K 31/497

(57) ABSTRACT

This disclosure provides crystalline forms of an androgen receptor modulator, and methods of making and using these forms.

### RELATED APPLICATIONS

This application claims priority to U.S. Provisional Application No. 63/625,470, filed Jan. 26, 2024, the content of which is hereby incorporated in its entirety.

### BACKGROUND

Prostate cancer is the second leading cause of male cancer-related death in Western countries (Damber, J. E. and Aus, G. Lancet (2008) 371:1710-1721). Numerous studies have shown that the androgen receptor (AR) is central not only to the development of prostate cancer, but also the progression of the disease to the castration resistance state (Taplin, M. E. et al. J. Clin. Oncol. (2003) 21:2673-8; and Tilley, W. D. et al. Cancer Res. (1994) 54:4096-4102). Thus, effective inhibition of human AR remains one of the most effective therapeutic approaches to the treatment of advanced, metastatic prostate cancer.

Spinal and Bulbar Muscular Atrophy (SBMA) or Kennedy's disease, is an x-linked recessively inherited neuromuscular disorder. The main symptoms are weakness and atrophy of bulbar and extremity muscles due to lower motor degeneration in the brainstem and spinal cord together with primary muscle involvement. At onset, patients often manifest limb weakness, cramps, tremor, and contraction fasciculations, especially noticeable in face and tongue. Dysarthria is also common with hypernasality, laryngospasm, and swallowing dysfunctions which frequently result in aspiration pneumonia as the disease progresses. More than half of patients die from respiratory infections diseases. The disease is caused by CAG expansions in exon 1 of the androgen receptor (AR), and androgen is required for the onset. At present there is no treatment for Kennedy's disease.

### SUMMARY

Provided herein are crystalline forms useful for the treatment of diseases such as Kennedy's disease in a subject in need thereof. In a particular aspect, provided herein are crystalline forms of 3-(5-(2-hydroxy-2-methylpropoxy)-6-methylpyrazin-2-yl)-1H-indole-7-carbonitrile free base having the formula:

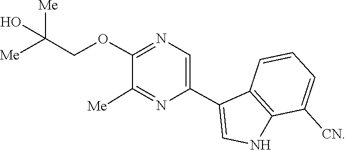 [SMILES: Cc1nc(-c2c[nH]c3c(C#N)cccc23)cnc1OCC(C)(C)O]

In another aspect, provided herein is a method of treating Kennedy's disease in a subject in need thereof comprising administering to the subject a crystalline form of 3-(5-(2-hydroxy-2-methylpropoxy)-6-methylpyrazin-2-yl)-1H-indole-7-carbonitrile free base.

Methods for preparing and analyzing Forms A-O are disclosed in International Application No. PCT/US2023/060619 (WO 2023/137420), the entire content of which is incorporated by reference herein.

### BRIEF DESCRIPTION OF THE DRAWINGS

FIG. 1A shows the XRPD diffractogram of crystalline 3-(5-(2-hydroxy-2-methylpropoxy)-6-methylpyrazin-2-yl)-1H-indole-7-carbonitrile anhydrate Form P

FIG. 1B shows the XRPD diffractogram of crystalline 3-(5-(2-hydroxy-2-methylpropoxy)-6-methylpyrazin-2-yl)-1H-indole-7-carbonitrile anhydrate milled Form P

FIG. 2A shows the DSC thermogram of crystalline 3-(5-(2-hydroxy-2-methylpropoxy)-6-methylpyrazin-2-yl)-1H-indole-7-carbonitrile anhydrate Form P.

FIG. 2B shows the DSC thermogram of crystalline 3-(5-(2-hydroxy-2-methylpropoxy)-6-methylpyrazin-2-yl)-1H-indole-7-carbonitrile anhydrate milled Form P.

FIG. 3A shows the TGA thermogram of 3-(5-(2-hydroxy-2-methylpropoxy)-6-methylpyrazin-2-yl)-1H-indole-7-carbonitrile anhydrate Form P.

FIG. 3B shows the TGA thermogram of 3-(5-(2-hydroxy-2-methylpropoxy)-6-methylpyrazin-2-yl)-1H-indole-7-carbonitrile anhydrate milled Form P.

FIG. 4 shows the ¹H NMR spectrum of 3-(5-(2-hydroxy-2-methylpropoxy)-6-methylpyrazin-2-yl)-1H-indole-7-carbonitrile anhydrate Form P.

FIG. 5 is a schematic diagram of the interconversion relation of polymorphs of 3-(5-(2-hydroxy-2-methylpropoxy)-6-methylpyrazin-2-yl)-1H-indole-7-carbonitrile.

FIG. 6 shows the XRPD diffractogram of 3-(5-(2-hydroxy-2-methylpropoxy)-6-methylpyrazin-2-yl)-1H-indole-7-carbonitrile Form Q.

FIG. 7 shows the XRPD diffractogram of 3-(5-(2-hydroxy-2-methylpropoxy)-6-methylpyrazin-2-yl)-1H-indole-7-carbonitrile hydrate Form R.

FIG. 8 shows the XRPD diffractogram of 3-(5-(2-hydroxy-2-methylpropoxy)-6-methylpyrazin-2-yl)-1H-indole-7-carbonitrile Form S.

FIG. 9 shows the XRPD diffractogram of 3-(5-(2-hydroxy-2-methylpropoxy)-6-methylpyrazin-2-yl)-1H-indole-7-carbonitrile Form T.

FIG. 10 shows the XRPD diffractogram of 3-(5-(2-hydroxy-2-methylpropoxy)-6-methylpyrazin-2-yl)-1H-indole-7-carbonitrile hydrate Form U.

FIG. 11 shows the XRPD diffractogram of 3-(5-(2-hydroxy-2-methylpropoxy)-6-methylpyrazin-2-yl)-1H-indole-7-carbonitrile Form V.

FIG. 12 shows the XRPD diffractogram of 3-(5-(2-hydroxy-2-methylpropoxy)-6-methylpyrazin-2-yl)-1H-indole-7-carbonitrile Form W.

FIG. 13 shows the XRPD diffractogram of 3-(5-(2-hydroxy-2-methylpropoxy)-6-methylpyrazin-2-yl)-1H-indole-7-carbonitrile hydrate Form X.

FIG. 14 shows the XRPD diffractogram of 3-(5-(2-hydroxy-2-methylpropoxy)-6-methylpyrazin-2-yl)-1H-indole-7-carbonitrile Form Y.

FIG. 15 shows the XRPD diffractogram of amorphous 3-(5-(2-hydroxy-2-methylpropoxy)-6-methylpyrazin-2-yl)-1H-indole-7-carbonitrile.

FIG. 16 shows the ORTEP diagram of the single-crystal X-ray structure of Form P.

FIG. 17 show the overlayed comparison of the simulated XRPD and experimental XRPD data of Form P.

### DETAILED DESCRIPTION

The solid state of a compound can be important when the compound is used for pharmaceutical purposes. The physical properties of a compound can change from one solid form to another, which can affect the suitability of the form for pharmaceutical use. For example, a particular crystalline solid compound can overcome the disadvantage of other solid forms of the compound such as, e.g., instability and/or reduced purity.

Provided herein are solid, crystalline forms of 3-(5-(2-hydroxy-2-methylpropoxy)-6-methylpyrazin-2-yl)-1H-indole-7-carbonitrile:

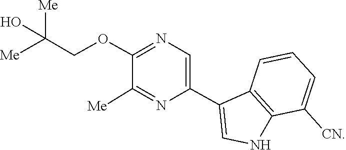 [SMILES: Cc1nc(-c2c[nH]c3c(C#N)cccc23)cnc1OCC(C)(C)O]

This compound is useful for the treatment of a variety of indications, including Kennedy's disease, in a subject.

In particular, provided herein are crystalline forms of 3-(5-(2-hydroxy-2-methylpropoxy)-6-methylpyrazin-2-yl)-1H-indole-7-carbonitrile or a solvate or hydrate thereof.

The compound 3-(5-(2-hydroxy-2-methylpropoxy)-6-methylpyrazin-2-yl)-1H-indole-7-carbonitrile is disclosed in PCT Application No. PCT/US2021/042355 and U.S. patent application Ser. No. 17/380,736, the entire contents of which are incorporated herein by reference.

The crystalline forms provided herein can be characterized by X-ray powder diffraction (XRPD), differential scanning calorimetry (DSC), and thermogravimetric analysis (TGA).

### Definitions

Listed below are definitions of various terms used to describe the crystalline forms provided herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which the compound and its crystalline forms belong. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “pharmaceutically acceptable carrier” refers to a pharmaceutically acceptable material, composition, or carrier, such as a liquid or solid filler, stabilizer, dispersing agent, suspending agent, diluent, excipient, thickening agent, solvent or encapsulating material, involved in carrying or transporting a compound useful within the present disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient.

Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound provided herein and are physiologically acceptable to the patient. Supplementary active compounds may also be incorporated into the compositions. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, PA), which is incorporated herein by reference.

As used herein, the phrases “therapeutically effective dose” and “therapeutically effective amount” refer to an amount of a compound that prevents the onset, alleviates the symptoms, stops the progression of a disease, or results in another desired biological outcome such as, e.g., improved clinical signs.

The term “treat,” “treated,” “treating,” or “treatment” includes the diminishment or alleviation of at least one symptom associated or caused by the state, disorder or disease being treated. In certain embodiments, the treatment comprises bringing into contact with the opioid receptor an effective amount of the compound provided herein for conditions related to androgen receptors.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. In an embodiment, the patient, subject, or individual is human.

The term “administering” or “administration” and the like, refers to providing a therapeutic agent, such as a crystalline form disclosed herein, to the subject in need of treatment. In an embodiment, the subject is a mammal. In another embodiment, the subject is a human.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of +10%, including ±5%, 1%, and ±0.1% from the specified value, as such variations are appropriate to perform the disclosed methods.

Solvents can be broadly classified into polar (hydrophilic) and nonpolar (lipophilic). The polarity can be measured as the dielectric constant or the dipole moment of a compound.

Nonpolar solvents include alkanes such as pentane (n-pentane), hexane (n-hexane), heptane (n-heptane), and cyclohexane. Additional examples of nonpolar solvents include benzene, toluene, chloroform, and diethyl ether, petroleum ether.

Examples of polar solvents include pyridine, isopropyl acetate (IPAc), dichloromethane (DCM), acetone, dimethylformamide (DMF), t-butyl alcohol, dimethylsulfoxide (DMSO), acetonitrile, isopropanol, benzyl alcohol, acetic acid, ethanol, methanol, and water.

An aprotic solvent is an organic solvent that does not contain an O—H or N—H bond; or does not exchange protons with a substance dissolved in it. Examples of aprotic solvents include dimethyl sulfoxide (DMSO), isopropyl acetate (IPAc), dimethylformamide (DMF), dichloromethane (DCM), acetonitrile, acetone, methyl ethyl ketone (MEK), methyl t-butyl ether (MBTE), 2-methyltetrahydrofuran (2-MeTHF), 1,4-dioxane, ethyl acetate, tetrahydrofuran (THF), heptane (n-heptane), methylcyclohexane, and toluene. Additional non-limiting examples include N-methylpyrrolidone (NMP), pyridine, piperidine, dimethyl ether, and methyl dodecyl sulfoxide.

Protic solvents are those solvents that contain an O—H or N—H bond. Typical protic solvents that may be used herein include various types of glycols, e.g., tripropylene glycol methyl ether, dipropylene glycol, and propylene glycol. Examples of other protic solvents include water, ammonia, acetic acid, formic acid, and alcohols such as methanol, ethanol, isopropanol, propanol, and butanol.

### Characterization of Crystalline Forms

In certain embodiments, the crystalline forms described herein are identifiable on the basis of characteristic peaks in an X-ray powder diffraction analysis. X-ray powder diffraction (XRPD) is a scientific technique using X-ray, neutron, or electron diffraction on powder, microcrystalline, or other solid materials for structural characterization of solid materials. A description of the methods used to obtain certain XRPD diffractograms in connection with the crystalline forms provided herein can be found in the Examples below. In an embodiment, the X-ray powder diffraction data provided herein is obtained by a method utilizing Cu Kα radiation.

In an aspect, provided herein is a crystalline form of 3-(5-(2-hydroxy-2-methylpropoxy)-6-methylpyrazin-2-yl)-1H-indole-7-carbonitrile, wherein the crystalline form is characterized by an XRPD diffractogram having peaks expressed in degrees-2-theta at angles (±0.2 degrees) of 23.7, 11.1, and 14.9.

In an embodiment, the crystalline form is characterized by an XRPD diffractogram having peaks expressed in degrees-2-theta at angles (±0.2 degrees) of 23.7, 11.1, 14.9, and 19.7.

In still another embodiment, the crystalline form is characterized by an XRPD diffractogram having peaks expressed in degrees-2-theta at angles (±0.2 degrees) of 23.7, 11.1, 14.9, 19.7, and 9.9.

In an embodiment, the crystalline form is characterized by an XRPD diffractogram having peaks expressed in degrees-2-theta at angles (±0.2 degrees) of 23.7, 11.1, 14.9, 19.7, 9.9, 29.3, and 24.7.

In another embodiment, the crystalline form is characterized by an XRPD diffractogram having peaks expressed in degrees-2-theta at angles (±0.2 degrees) of 23.7, 11.1, 14.9, 19.7, 9.9, 29.3, 24.7, 21.4, 17.1, 18.4, and 20.6.

In yet another embodiment, the crystalline form is characterized by an XRPD diffractogram having peaks expressed in degrees-2-theta at angles (±0.2 degrees) of 23.7, 11.1, 14.9, 19.7, 9.9, 29.3, 24.7, 21.4, 17.1, 18.4, 20.6, 16.9, and 24.2.

In an embodiment, the crystalline form is characterized by an XRPD diffractogram having peaks selected from Table 1 (Form P).

TABLE 1

| Angle | d Value | Rel. Intensity |
|---|---|---|
| 8.052° | 10.97125 Å | 1.7% |
| 9.929° | 8.90104 Å | 36.3% |
| 11.050° | 8.00089 Å | 9.9% |
| 11.617° | 7.61107 Å | 83.5% |
| 14.145° | 6.25611 Å | 1.0% |
| 14.947° | 5.92249 Å | 77.5% |
| 15.505° | 5.71036 Å | 6.1% |
| 16.906° | 5.24011 Å | 11.9% |
| 17.128° | 5.17280 Å | 8.6% |
| 17.997° | 4.92495 Å | 0.5% |
| 18.348° | 4.83141 Å | 19.9% |
| 18.884° | 4.69566 Å | 6.1% |
| 19.699° | 4.50317 Å | 50.9% |
| 20.259° | 4.37977 Å | 9.9% |
| 20.552° | 4.31804 Å | 14.8% |
| 21.365° | 4.15553 Å | 22.1% |
| 22.156° | 4.00899 Å | 0.7% |
| 22.695° | 3.91496 Å | 3.1% |
| 23.650° | 3.75894 Å | 100.0% |
| 24.219° | 3.67199 Å | 10.2% |
| 24.683° | 3.60396 Å | 25.7% |
| 24.928° | 3.56908 Å | 8.7% |
| 25.514° | 3.48838 Å | 21.0% |
| 26.266° | 3.39016 Å | 1.2% |
| 27.461° | 3.24540 Å | 8.2% |
| 28.304° | 3.15062 Å | 32.4% |
| 29.022° | 3.07421 Å | 1.8% |
| 29.339° | 3.04179 Å | 5.9% |
| 30.352° | 2.94247 Å | 4.9% |
| 30.789° | 2.90176 Å | 0.5% |
| 31.059° | 2.87712 Å | 3.5% |
| 32.485° | 2.75397 Å | 8.4% |
| 33.003° | 2.71197 Å | 4.0% |
| 33.443° | 2.67726 Å | 1.0% |
| 34.127° | 2.62514 Å | 1.6% |
| 34.610° | 2.58961 Å | 1.1% |
| 34.870° | 2.57087 Å | 1.9% |
| 35.367° | 2.53586 Å | 5.0% |
| 35.780° | 2.50760 Å | 3.1% |
| 36.252° | 2.47597 Å | 1.4% |
| 36.645° | 2.45031 Å | 3.4% |
| 37.286° | 2.40969 Å | 0.4% |
| 38.160° | 2.35645 Å | 1.2% |
| 38.952° | 2.31038 Å | 6.3% |

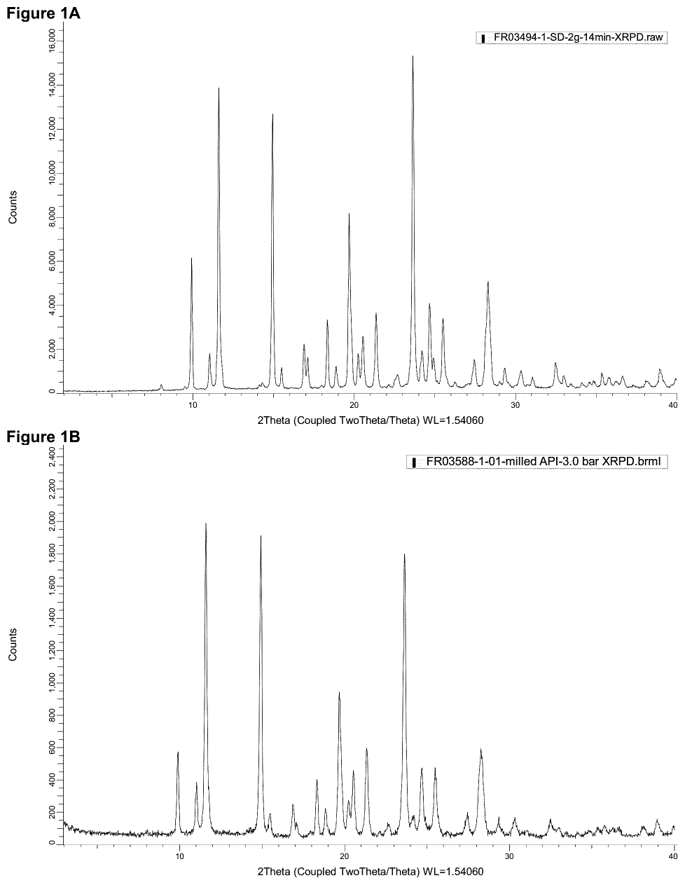

In another embodiment, the crystalline form has the XRPD diffractogram substantially as depicted in FIG. 1A. In another embodiment, the crystalline form has the XRPD diffractogram substantially as depicted in FIG. 1B.

In an embodiment, the crystalline form has a DSC thermogram characterized by an endotherm with an onset temperature range 168.5-171.5° C. In another embodiment, the crystalline form has a DSC thermogram characterized by an endotherm with an onset temperature range of 178.5-180.5° C.

In another embodiment, the crystalline form has a DSC thermogram characterized by an initial endotherm with an onset temperature of about 170.5° C., followed by a second endotherm with an onset temperature of about 179.9° C.

In yet another embodiment, the crystalline form has a DSC thermogram characterized by an initial endotherm with an onset temperature of 169.5° C., followed by a second endotherm at with an onset temperature of about 179.7° C.

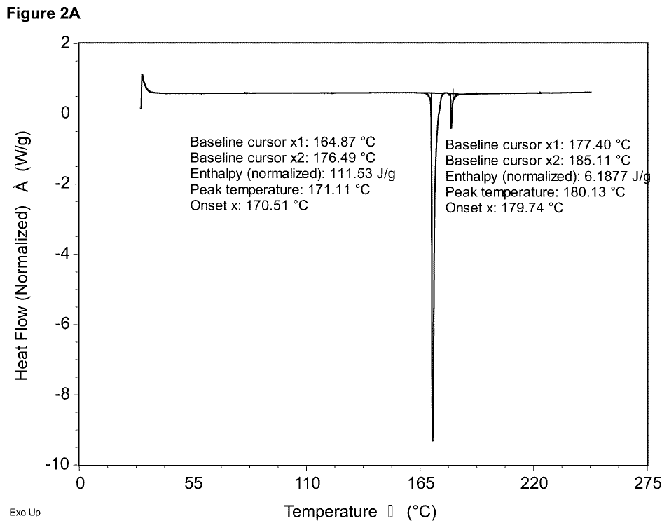

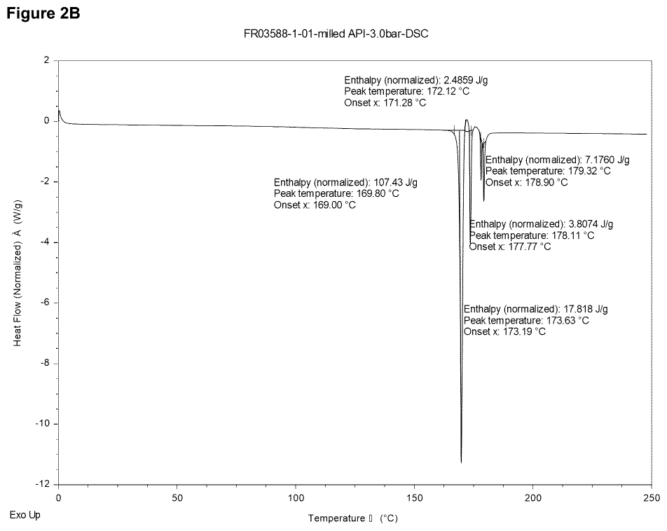

In still another embodiment, the crystalline form has a DSC thermogram substantially as depicted in FIG. 2A.

### Methods of Treatment

Provided herein are methods for the treatment of a disease comprising administering a crystalline form of 3-(5-(2-hydroxy-2-methylpropoxy)-6-methylpyrazin-2-yl)-1H-indole-7-carbonitrile, or a pharmaceutical composition comprising the crystalline form and a pharmaceutically acceptable carrier.

In an aspect, provided herein is a method of treating a neurodegenerative disorder in a subject in need thereof comprising administering to the subject the crystalline form, or a pharmaceutical composition comprising the crystalline form and a pharmaceutically acceptable carrier.

In an embodiment, the neurodegenerative disorder is spinal bulbar muscular atrophy (SBMA).

In an embodiment of the methods, the crystalline form is anhydrous.

In another embodiment of the method, the crystalline form is characterized by an XRPD diffractogram having peaks expressed in degrees-2-theta at angles (±0.2 degrees) of 23.7, 11.1, and 14.9.

In yet another embodiment of the method, the crystalline form is characterized by an XRPD diffractogram having peaks expressed in degrees-2-theta at angles (±0.2 degrees) of 23.7, 11.1, 14.9, and 9.9.

In still another embodiment of the method, the crystalline form is characterized by an XRPD diffractogram having peaks expressed in degrees-2-theta at angles (±0.2 degrees) of 23.7, 11.1, 14.9, 9.9, and 19.7.

In another embodiment of the method, the crystalline form is characterized by an XRPD diffractogram having peaks expressed in degrees-2-theta at angles (±0.2 degrees) of 23.7, 11.1, 14.9, 9.9, 19.7, 29.3, and 24.7.

In yet another embodiment of the method, the crystalline form is characterized by an XRPD diffractogram having peaks expressed in degrees-2-theta at angles (±0.2 degrees) of 23.7, 11.1, 14.9, 9.9, 19.7, 29.3, 24.7, 21.4, and 17.1. In still another embodiment of the method, the crystalline form is characterized by an XRPD diffractogram having peaks expressed in degrees-2-theta at angles (±0.2 degrees) of 23.7, 11.1, 14.9, 9.9, 19.7, 29.3, 24.7, 21.4, 17.1, 18.4, and 20.6.

In another embodiment of the method, the crystalline form is characterized by an XRPD diffractogram having peaks expressed in degrees-2-theta at angles (±0.2 degrees) of 23.7, 11.1, 14.9, 9.9, 19.7, 29.3, 24.7, 21.4, 17.1, 18.4, 20.6, 16.9, and 24.2.

In another embodiment of the method, the crystalline form is characterized by an XRPD diffractogram having peaks selected from Table 1.

In another aspect, provided herein is a method of modulating androgen receptor (AR) activity in a subject in need thereof comprising administering to the subject a crystalline form of the present disclosure, or a pharmaceutical composition comprising the crystalline form and a pharmaceutically acceptable carrier.

In an embodiment, the androgen receptor (AR) undergoes allosteric modulation. In another embodiment, modulating androgen receptor (AR) activity treats spinal bulbar muscular atrophy (SBMA) in the subject.

In another embodiment, the crystalline form selectively binds to the binding function-3 (BF3) domain of the androgen receptor.

In yet another aspect, provided herein is a method of treating spinal bulbar muscular atrophy (SBMA) in a subject in need thereof comprising administering to the subject a crystalline form of the present disclosure.

In still another aspect, provided herein is a method of treating spinal bulbar muscular atrophy (SBMA) in a subject in need thereof comprising administering to the subject a pharmaceutical composition comprising a crystalline form of the present disclosure and a pharmaceutically acceptable carrier.

In yet another aspect, provided herein is a method of treating cancer in a subject in need thereof comprising administering to the subject the crystalline form of the present disclosure, or a pharmaceutical composition comprising the crystalline form and a pharmaceutically acceptable carrier.

In an embodiment, the cancer is selected from hematological cancers, sarcomas, lung cancers, gastrointestinal cancers, genitourinary tract cancers, liver cancers, bone cancers, nervous system cancers, gynecological cancers, and skin cancers.

In another embodiment, the lung cancer is selected from non-small cell lung cancer (NSCLC), small cell lung cancer, bronchogenic carcinoma, squamous cell bronchogenic carcinoma, undifferentiated small cell bronchogenic carcinoma, undifferentiated large cell bronchogenic carcinoma, adenocarcinoma, bronchogenic carcinoma, alveolar carcinoma, bronchiolar carcinoma, bronchial adenoma, chondromatous hamartoma, mesothelioma, pavicellular and non-pavicellular carcinoma, bronchial adenoma, and pleuropulmonary blastoma.

In yet another embodiment, the lung cancer is non-small cell lung cancer (NSCLC). In still another embodiment, the lung cancer is adenocarcinoma.

In an embodiment, the gastrointestinal cancer is selected from esophagus squamous cell carcinoma, esophagus adenocarcinoma, esophagus leiomyosarcoma, esophagus lymphoma, stomach carcinoma, stomach lymphoma, stomach leiomyosarcoma, exocrine pancreatic carcinoma, pancreatic ductal adenocarcinoma, pancreatic insulinoma, pancreatic glucagonoma, pancreatic gastrinoma, pancreatic carcinoid tumors, pancreatic vipoma, small bowel adenocarcinoma, small bowel lymphoma, small bowel carcinoid tumors, Kaposi's sarcoma, small bowel leiomyoma, small bowel hemangioma, small bowel lipoma, small bowel neurofibroma, small bowel fibroma, large bowel adenocarcinoma, large bowel tubular adenoma, large bowel villous adenoma, large bowel hamartoma, large bowel leiomyoma, colorectal cancer, gall bladder cancer, and anal cancer.

In an embodiment, the gastrointestinal cancer is colorectal cancer.

In another embodiment, the cancer is a carcinoma. In yet another embodiment, the carcinoma is selected from pancreatic carcinoma, colorectal carcinoma, lung carcinoma, bladder carcinoma, gastric carcinoma, esophageal carcinoma, breast carcinoma, head and neck carcinoma, cervical skin carcinoma, and thyroid carcinoma.

In still another embodiment, the cancer is a hematopoietic malignancy. In an embodiment, the hematopoietic malignancy is selected from multiple myeloma, acute myelogenous leukemia, and myeloproliferative neoplasms.

In another embodiment, the cancer is a neoplasm. In yet another embodiment, the neoplasm is glioblastoma or sarcomas.

In an embodiment, the cancer is selected from the group consisting of hematological cancers, sarcomas, lung cancers, gastrointestinal cancers, genitourinary tract cancers, liver cancers, bone cancers, nervous system cancers, gynecological cancers, and skin cancers.

In an embodiment, the cancer is selected from the group consisting of pancreatic cancer, cervical cancer, colon cancer, ovarian cancer, breast cancer, pancreatic cancer, carcinoma, and adenocarcinoma.

In another embodiment, the cancer is pancreatic cancer. In yet another embodiment, the cancer is a solid tumor.

In one embodiment of the methods described herein, the subject is human.

### Processes for Preparing Form P

In an aspect, provided herein is a process for preparing the crystalline form of combining 3-(5-(2-hydroxy-2-methylpropoxy)-6-methylpyrazin-2-yl)-1H-indole-7-carbonitrile comprising:
a) combining 3-(5-(2-hydroxy-2-methylpropoxy)-6-methylpyrazin-2-yl)-1H-indole-7-carbonitrile with a first polar, aprotic solvent to form a suspension;
b) stirring the suspension at between 45° C. and 55° C. to form a solution;
c) cooling the solution to between 20° C. and 30° C. to form a suspension;
d) adding to the suspension seeds of crystalline 3-(5-(2-hydroxy-2-methylpropoxy)-6-methylpyrazin-2-yl)-1H-indole-7-carbonitrile;
e) stirring the suspension at about 5° C.;
f) adding a second non-polar, aprotic solvent;
g) stirring the suspension at about 5° C.;
h) collecting the solids by crystalline form of 3-(5-(2-hydroxy-2-methylpropoxy)-6-methylpyrazin-2-yl)-1H-indole-7-carbonitrile.

In an embodiment, the polar, aprotic solvent in step a) is 2-methyltetrahydrofuran.

In another embodiment, the non-polar, aprotic solvent in step f) is heptane.

In an embodiment of the foregoing processes, the starting material is amorphous.

In another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, and 14.9. In yet another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, 14.9, and 9.9.

In still another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, 14.9, 9.9, and 19.7.

In another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, 14.9, 9.9, 19.7, 29.3, 24.7, 21.4, 17.1, 18.4, 20.6, 16.9, and 24.2.

In an embodiment, the seeds of crystalline 3-(5-(2-hydroxy-2-methylpropoxy)-6-methylpyrazin-2-yl)-1H-indole-7-carbonitrile in step b) are characterized by an XRPD diffractogram having peaks selected from Table 1 (Form P).

In another embodiment, the crystalline form of 3-(5-(2-hydroxy-2-methylpropoxy)-6-methylpyrazin-2-yl)-1H-indole-7-carbonitrile in step h) is filtered by centrifugation and dried at about 25° C. under vacuum.

In an aspect, provided herein is a process for preparing the crystalline form of combining 3-(5-(2-hydroxy-2-methylpropoxy)-6-methylpyrazin-2-yl)-1H-indole-7-carbonitrile comprising:
a) combining 3-(5-(2-hydroxy-2-methylpropoxy)-6-methylpyrazin-2-yl)-1H-indole-7-carbonitrile with a polar, protic solvent to form a suspension;
b) stirring the suspension at between 35° C. and 40° C.; and
c) collecting crystalline form of 3-(5-(2-hydroxy-2-methylpropoxy)-6-methylpyrazin-2-yl)-1H-indole-7-carbonitrile by centrifugation.

In an embodiment of the foregoing processes, the starting material is crystalline Form C.

In another embodiment, wherein Form C is defined as an anhydrous crystalline polymorphic form of 3-(5-(2-hydroxy-2-methylpropoxy)-6-methylpyrazin-2-yl)-1H-indole-7-carbonitrile.

In another embodiment, the crystalline Form C is characterized by an XRPD diffractogram having peaks expressed in degrees-2-theta at angles (±0.2 degrees) of 23.8, 20.7, and 11.7.

In yet another embodiment, the crystalline Form C is characterized by an XRPD diffractogram having peaks expressed in degrees-2-theta at angles (±0.2 degrees) of 23.8, 20.7, 11.7, and 24.6.

In still another embodiment, the crystalline Form C is characterized by an XRPD diffractogram having peaks expressed in degrees-2-theta at angles (±0.2 degrees) of 23.8, 20.7, 11.7, 24.6, and 15.8.

In another embodiment, the crystalline Form C is characterized by an XRPD diffractogram having peaks expressed in degrees-2-theta at angles (±0.2 degrees) of 23.8, 20.7, 11.7, 24.6, 15.8, 11.5, 12.2, 12.5, 16.3, 22.5, 22.9, and 30.0.

In embodiment, the polar, protic solvent in step a) is aqueous.

In still another embodiment the aqueous solvent is an acidic buffer with a pH of approximately 1 In an embodiment the acidic buffer is a pH 1.2 HCl buffer solution.

In yet another embodiment, the crystalline form is dried at between 35° C. and 40° C. under vacuum.

In an aspect, provided herein is a process for preparing the crystalline form of combining 3-(5-(2-hydroxy-2-methylpropoxy)-6-methylpyrazin-2-yl)-1H-indole-7-carbonitrile comprising:
a) combining 3-(5-(2-hydroxy-2-methylpropoxy)-6-methylpyrazin-2-yl)-1H-indole-7-carbonitrile with a polar, aprotic solvent to form a suspension;
b) stirring the suspension at between 45° C. and 55° C.;
c) adding to the mixture seeds of crystalline 3-(5-(2-hydroxy-2-methylpropoxy)-6-methylpyrazin-2-yl)-1H-indole-7-carbonitrile;
e) re-equilibrating the solids in the polar, aprotic solvent under a temperature cycle between about 5° C. to about 50° C.; and
f) collecting the crystalline form of 3-(5-(2-hydroxy-2-methylpropoxy)-6-methylpyrazin-2-yl)-1H-indole-7-carbonitrile by centrifugation.

In an embodiment, the polar, aprotic solvent in step a) is acetone.

In another embodiment, the polar, aprotic solvent in step e) is acetone.

In an embodiment of the foregoing processes, the starting material is crystalline Form C, wherein Form C is defined above.

In another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, and 14.9.

In yet another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, 14.9, and 9.9.

In still another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, 14.9, 9.9, and 19.7.

In another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, 14.9, 9.9, 19.7, 29.3, 24.7, 21.4, 17.1, 18.4, 20.6, 16.9, and 24.2.

In an embodiment, the seeds of crystalline 3-(5-(2-hydroxy-2-methylpropoxy)-6-methylpyrazin-2-yl)-1H-indole-7-carbonitrile in step c) are characterized by an XRPD diffractogram having peaks selected from Table 1 (Form P).

In an aspect, provided herein is a process for preparing the crystalline form of 3-(5-(2-hydroxy-2-methylpropoxy)-6-methylpyrazin-2-yl)-1H-indole-7-carbonitrile comprising:
a) combining 3-(5-(2-hydroxy-2-methylpropoxy)-6-methylpyrazin-2-yl)-1H-indole-7-carbonitrile with a polar, aprotic solvent to form a suspension;
b) stirring the suspension at between 45° C. and 55° C.;
c) adding to the suspension seeds of crystalline 3-(5-(2-hydroxy-2-methylpropoxy)-6-methylpyrazin-2-yl)-1H-indole-7-carbonitrile;
e) re-equilibrating the solids in the polar, aprotic solvent under a temperature cycle between about 5° C. to about 50° C.;
f) adding to the suspension a bulk of crystalline 3-(5-(2-hydroxy-2-methylpropoxy)-6-methylpyrazin-2-yl)-1H-indole-7-carbonitrile dissolved in a polar, aprotic solvent;
g) re-equilibrating the solids in the polar, aprotic solvent under a temperature cycle between about 5° C. to about 50° C.;
h) collecting the crystalline form of 3-(5-(2-hydroxy-2-methylpropoxy)-6-methylpyrazin-2-yl)-1H-indole-7-carbonitrile.

In an embodiment, the polar, aprotic solvent in step a) is acetone.

In another embodiment, the polar, aprotic solvent in step e) is acetone

In yet another embodiment, the polar, aprotic solvent in step f) is acetone

In still another embodiment, the polar, aprotic solvent in step g) is acetone

In an embodiment of the foregoing processes, the starting material is crystalline Form C, wherein Form C is defined above.

In another embodiment of the foregoing processes, 3-(5-(2-hydroxy-2-methylpropoxy)-6-methylpyrazin-2-yl)-1H-indole-7-carbonitrile in step f) is crystalline Form C.

In another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, and 14.9.

In yet another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, 14.9, and 9.9.

In still another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, 14.9, 9.9, and 19.7.

In another embodiment, the seeds of crystalline 3-(5-(2-hydroxy-2-methylpropoxy)-6-methylpyrazin-2-yl)-1H-indole-7-carbonitrile in step d) are characterized by an XRPD diffractogram having peaks expressed in degrees-2-theta at angles (±0.2 degrees) of 23.7, 11.1, 14.9, 9.9, 19.7, 29.3, 24.7, 21.4, 17.1, 18.4, 20.6, 16.9, and 24.2.

In an embodiment, the seeds of crystalline 3-(5-(2-hydroxy-2-methylpropoxy)-6-methylpyrazin-2-yl)-1H-indole-7-carbonitrile in step c) are characterized by an XRPD diffractogram having peaks selected from Table 1 (Form P).

In another embodiment, the final collected crystalline form is dried at about 25° C. under vacuum.

In another aspect, provided herein is a process for preparing the crystalline form of any one of claims 1-11 comprising dissolving 3-(5-(2-hydroxy-2-methylpropoxy)-6-methylpyrazin-2-yl)-1H-indole-7-carbonitrile in a solvent system to form a suspension, heating the suspension to between 45° C. and 55° C., passing the suspension through a filter to isolate a solution, cooling the solution to form a slurry, and filtering the slurry to isolate the crystalline form of 3-(5-(2-hydroxy-2-methylpropoxy)-6-methylpyrazin-2-yl)-1H-indole-7-carbonitrile.

In an embodiment, the solvent system is selected from the group consisting of acetone, ethyl acetate, isopropyl acetate, methanol, methyl ethyl ketone (MEK), 2-methyltetrahydrofuran (2-MeTHF), and water or a combination thereof.

In another embodiment, the solvent system is acetone/water. In yet another embodiment, the solvent system is about 30% acetone and 70% water. In still another embodiment, the solvent system is methanol/water. In an embodiment, the solvent system is about 40% methanol and 60% water.

In yet another aspect, provided herein is a process for preparing the crystalline form of any one of claims 1-11 comprising dissolving 3-(5-(2-hydroxy-2-methylpropoxy)-6-methylpyrazin-2-yl)-1H-indole-7-carbonitrile in a first solvent, adding a second solvent to form a slurry, stirring the slurry for 8 days, and filtering the slurry to isolate the crystalline form of 3-(5-(2-hydroxy-2-methylpropoxy)-6-methylpyrazin-2-yl)-1H-indole-7-carbonitrile.

In an embodiment, the first solvent is selected from the group consisting of acetone, dichloromethane, dimethyl sulfoxide, ethyl acetate, methanol, 2-methyltetrahydrofuran (2-MeTHF), and tetrahydrofuran, or a combination thereof.

In another embodiment, the first solvent is acetone. In yet another embodiment, the first solvent is dimethyl sulfoxide. In still another embodiment, the first solvent is methanol. In an embodiment, the first solvent is tetrahydrofuran. In another embodiment, the first solvent is ethyl acetate. In yet another embodiment, the first solvent is dichloromethane. In still another embodiment, the first solvent is 2-methyltetrahydrofuran.

In an embodiment, the second solvent is water. In another embodiment, the second solvent is heptane. In yet another embodiment, the second solvent is methyl tert-butyl ether.

In an aspect, provided herein is a process for preparing a crystalline form of 3-(5-(2-hydroxy-2-methylpropoxy)-6-methylpyrazin-2-yl)-1H-indole-7-carbonitrile comprising:
a) combining 3-(5-(2-hydroxy-2-methylpropoxy)-6-methylpyrazin-2-yl)-1H-indole-7-carbonitrile with the solvent methyl ethyl ketone (MEK) to form a suspension;
b) filtering the suspension to obtain a solution;
c) evaporating the solvent;
d) collecting the crystalline form of 3-(5-(2-hydroxy-2-methylpropoxy)-6-methylpyrazin-2-yl)-1H-indole-7-carbonitrile by centrifugation.

In another aspect, provided herein is a process for preparing a crystalline form of 3-(5-(2-hydroxy-2-methylpropoxy)-6-methylpyrazin-2-yl)-1H-indole-7-carbonitrile comprising:
a) dissolving 3-(5-(2-hydroxy-2-methylpropoxy)-6-methylpyrazin-2-yl)-1H-indole-7-carbonitrile with a solvent to form a suspension;
b) stirring the suspension at between 45° C. and 55° C.;
c) filtering the suspension to obtain a solution;
c) cooling the solution to between 0° C. and 10° C.,
d) collecting the crystalline form of 3-(5-(2-hydroxy-2-methylpropoxy)-6-methylpyrazin-2-yl)-1H-indole-7-carbonitrile by centrifugation.

In an embodiment, the first solvent is selected from the group consisting of acetone, ethyl acetate, isopropyl acetate (IPAc), methanol, methyl ethyl ketone (MEK), and 2-methyltetrahydrofuran (2-MeTHF), or a combination thereof.

In another embodiment, the second solvent is selected from the group consisting of heptane and water, or a combination thereof.

In yet another embodiment, the first solvent is acetone, and the second solvent is water.

In still another embodiment, the first solvent is methanol, and the second solvent is water.

In yet another embodiment, the first solvent is 2-methyltetrahydrofuran, and the second solvent is heptane.

In an embodiment, the rate of cooling in step c) is approximately 0.1° C./min.

### Pharmaceutical Compositions

In an aspect, provided herein is a pharmaceutical composition comprising a crystalline form provided herein and a pharmaceutically acceptable carrier.

In an embodiment, the pharmaceutical composition comprises a crystalline form that is substantially free from other crystalline forms.

In another embodiment, the crystalline form is characterized by an XRPD diffractogram having peaks expressed in degrees-2-theta at angles (±0.2 degrees) of 23.7, 11.1, and 14.9.

In yet another embodiment, the crystalline form is characterized by an XRPD diffractogram having peaks expressed in degrees-2-theta at angles (±0.2 degrees) of 23.7, 11.1, 14.9, and 9.9.

In another embodiment, the crystalline form is characterized by an XRPD diffractogram having peaks expressed in degrees-2-theta at angles (±0.2 degrees) of 23.7, 11.1, 14.9, 19.7, and 9.9.

In still another embodiment, the crystalline form is characterized by an XRPD diffractogram having peaks selected from Table 1. In another embodiment, the crystalline form has an XRPD diffractogram substantially as depicted in FIG. 1A. In another embodiment, the crystalline form has an XRPD diffractogram substantially as depicted in FIG. 1B.

The pharmaceutical compositions can be formulated for oral, intravenous, intramuscular, subcutaneous or parenteral administration for the therapeutic or prophylactic treatment of Kennedy's disease.

The pharmaceutical preparations disclosed herein can be prepared in accordance with standard procedures and are administered at dosages that are selected to reduce, prevent, or eliminate disease. See, for example, Remington's Pharmaceutical Sciences, Mack Publishing Company, Easton, PA and Goodman and Gilman's “The Pharmaceutical Basis of Therapeutics,” Pergamon Press, New York, NY, the contents of which are incorporated herein by reference, for a general description of the methods for administering various agents for human therapy.

The pharmaceutical compositions described herein can comprise a crystalline form disclosed herein in association with one or more nontoxic, pharmaceutically acceptable carriers and/or diluents and/or adjuvants and/or excipients.

For oral or parenteral administration, the crystalline form disclosed herein can be mixed with conventional pharmaceutical carriers and excipients and used in the form of tablets, capsules, elixirs, suspensions, syrups, wafers and the like. The compositions comprising a crystalline form disclosed herein can contain from about 0.1% to about 99% by weight of the active compound, such as from about 10% to about 30%.

For oral use, solid formulations such as tablets and capsules are useful. Sustained release or enterically coated preparations can also be devised. For pediatric and geriatric applications, one embodiment provides suspensions, syrups, and chewable tablets. For oral administration, the pharmaceutical compositions are in the form of, for example, a tablet, capsule, suspension, or liquid.

The pharmaceutical compositions can be made in the form of a dosage unit containing a therapeutically effective amount of the active ingredient. Examples of such dosage units are tablets and capsules. For therapeutic purposes, the tablets and capsules which can contain, in addition to the active ingredient, conventional carriers such as binding agents, fillers, lubricants, disintegrants, or acceptable wetting agents. Oral liquid preparations generally are in the form of aqueous or oily solutions, suspensions, emulsions, syrups, or elixirs.

The pharmaceutical compositions disclosed herein can be placed in a pharmaceutically acceptable carrier and are delivered to a recipient subject (e.g., a human) in accordance with known methods of drug delivery. In general, the methods of delivering the pharmaceutical compositions in vivo utilize art-recognized protocols for delivering the agent with the only substantial procedural modification being the substitution of a crystalline form of the present disclosure for the drugs in the art-recognized protocols.

### Administration/Dosage/Formulations

In another aspect, provided herein is a pharmaceutical composition comprising a crystalline form provided herein, together with a pharmaceutically acceptable carrier.

Actual dosage levels of the active ingredients in the pharmaceutical compositions discussed herein may be varied to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the crystalline form, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed crystalline form at levels lower than that required in order to achieve the desired therapeutic effect and gradually increase the dosage until the desired effect is achieved.

In particular embodiments, it is especially advantageous to formulate the crystalline form in dosage unit form for ease of administration and uniformity of dosage.

“Dosage unit form,” as used herein, refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the crystalline form disclosed herein are dictated by and directly dependent on (a) the unique characteristics of the disclosed compound and the particular therapeutic effect to be achieved, and (b) the limitations inherent in the art of compounding/formulating such a disclosed compound for the treatment of Kennedy's disease (SBMA) in a patient.

In one embodiment, the crystalline form provided herein is formulated using one or more pharmaceutically acceptable excipients or carriers. In one embodiment, the pharmaceutical compositions comprise a therapeutically effective amount of the disclosed crystalline form and a pharmaceutically acceptable carrier.

In some embodiments, the dose of a disclosed compound is from about 1 mg to about 1,000 mg. In some embodiments, a dose of the disclosed compound used in compositions described herein is less than about 1,000 mg, or less than about 800 mg, or less than about 600 mg, or less than about 500 mg, or less than about 300 mg, or less than about 200 mg, or less than about 100 mg, or less than about 50 mg, or less than about 20 mg, or less than about 10 mg. For example, a dose is about 10 mg, 20 mg, 25 mg, 30 mg, 40 mg, 50 mg, 60 mg, 70 mg, 80 mg, 90 mg, 100 mg, 120 mg, 140 mg, 160 mg, 180 mg, 200 mg, 220 mg, 240, 260 mg, 280 mg, 300 mg, 350 mg, 400 mg, 450 mg, 500 mg, 550 mg, or about 600 mg.

Routes of administration of any of the compositions disclosed herein include oral, nasal, rectal, intravaginal, parenteral, buccal, sublingual or topical. The compound for use provided herein may be formulated for administration by any suitable route, such as for oral or parenteral, for example, transdermal, transmucosal (e.g., sublingual, lingual, (trans)buccal, (trans)urethral, vaginal (e.g., trans- and peri-vaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions, and formulations for intravesical administration and the like. It should be understood that the formulations and compositions that would be useful in the present disclosure are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutical excipients that are suitable for the manufacture of tablets. Such excipients include, for example, an inert diluent such as lactose; granulating and disintegrating agents such as cornstarch; binding agents such as starch; and lubricating agents such as magnesium stearate. The tablets may be uncoated, or they may be coated by known techniques for elegance or to delay the release of the active ingredients. Formulations for oral use may also be presented as hard gelatin capsules wherein the active ingredient is mixed with an inert diluent.

For parenteral administration, the disclosed compound may be formulated for injection or infusion, for example, intravenous, intramuscular, or subcutaneous injection or infusion, or for administration in a bolus dose or continuous infusion. Suspensions, solutions, or emulsions in an oily or aqueous vehicle, optionally containing other formulatory agents such as suspending, stabilizing or dispersing agents may be used.

Those skilled in the art will recognize or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### Examples

The disclosure is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

A process for preparing amorphous 3-(5-(2-hydroxy-2-methylpropoxy)-6-methylpyrazin-2-yl)-1H-indole-7-carbonitrile and methods for measuring its biological activity is disclosed in PCT Application No. PCT/US2021/042355 and U.S. patent application Ser. No. 17/380,736, the entire contents of which are incorporated herein by reference.

### Synthesis of 3-(5-(2-hydroxy-2-methylpropoxy)-6-methylpyrazin-2-yl)-1H-indole-7-carbonitrile

Step 1: To a degassed mixture of 5-iodo-3-methyl-2-[2-methyl-2-(oxan-2-yloxy)propoxy]pyrazine (150 mg, 0.38 mmol) and tert-butyl 7-cyano-3-(4,4,5,5-tetramethyl-1,3,2-dioxaborolan-2-yl)indole-1-carboxylate (140 mg, 0.38 mmol) in dioxane (2 mL) and water (0.2 mL) were added potassium carbonate (105 mg, 0.76 mmol) and [1,1′-bis(diphenylphosphino)ferrocene]dichloropalladium(II) (27 mg, 0.04 mmol) at room temperature. The mixture was stirred at 100° C. for 2 hours. The reaction mixture was diluted using water and extracted with ethyl acetate. The organic combined layers were washed with brine, dried over anhydrous sodium sulfate, filtered and concentrated under vacuum. The residue was purified by flash column chromatography with 0-35% ethyl acetate in petroleum ether to afford 3-[6-methyl-5-[2-methyl-2-(oxan-2-yloxy)propoxy]pyrazin-2-yl]-1H-indole-7-carbonitrile (67 mg, 43%) as a white solid. MS m/z 407.1 [M+1]⁺.

Step 2: To a solution of 3-[6-methyl-5-[2-methyl-2-(oxan-2-yloxy)propoxy]pyrazin-2-yl]-1H-indole-7-carbonitrile (62 mg, 0.15 mmol) in dichloromethane (2.1 mL) was added trifluoroacetic acid (0.7 mL) at room temperature. The mixture was stirred at room temperature for 3 hours. The mixture was concentrated under vacuum. The residue was purified by reverse phase flash column chromatography with 10-80% acetonitrile in water (10 mmol/L ammonium bicarbonate) to afford 3-[5-(2-hydroxy-2-methylpropoxy)-6-methylpyrazin-2-yl]-1H-indole-7-carbonitrile (21.3 mg, 43%) as a white solid. ¹H NMR (400 MHz, DMSO-d₆) δ 12.35 (s, 1H), 8.73 (d, J=8.0 Hz, 1H), 8.56 (s, 1H), 8.19 (d, J=2.4 Hz, 1H), 7.69-7.67 (m, 1H), 7.28 (t, J=8.0 Hz, 1H), 4.70 (br, 1H), 4.10 (s, 2H), 2.54 (s, 3H), 1.24 (s, 6H); MS (ESI) calc'd for C₁₈H₁₈N₄O₂ [M+1]⁺, 323.1; found, 323.0.

### Biological Assay

LNCaP cells expressing ARR2PB-FireflyLuc and CMV-RenillaLuc were treated with indicated concentrations of the test compound, enzalutamide (negative control), or DHT (positive control)+/−0.5 nM DHT for 48 h at 37° C. Fluorescent signals were read with the ImageXpress Micro Confocal System. Remaining activity (antagonist mode) was calculated as % Remaining Activity=100×[(Readₛₐₘₚₗₑ−LCₐᵥₑ)/(HCₐᵥₑ−LCₐᵥₑ)] where HC is cells treated with 0.5 nM DHT only and LC is cells treated with 10 uM enzalutamide+0.5 nM DHT. Activation (agonist mode) was calculated as % Activation=100×[(ReadSample−LCₐᵥₑ)/(HCₐᵥₑ−LCₐᵥₑ)] where HC is cells treated with 1 uM DHT and LC is cells treated with DMSO. Dose response curves and IC₅₀ values were calculated using non-linear regression analysis in XLfit. 3-(5-(2-hydroxy-2-methylpropoxy)-6-methylpyrazin-2-yl)-1H-indole-7-carbonitrile was found to have an IC₅₀ value of 50 nM.

### Analytical Methods for Polymorph Studies

Unless otherwise indicated, X-ray powder diffraction (XRPD) was performed on a Bruker D8 Advance diffractometer in reflection mode using collimated Cu Kα radiation operating at 40 kV, 40 mA. Scans were run from 2-40 degrees 2-theta with a step size of 0.02 degrees and a scan time of 0.3 second per step.

Differential Scanning Calorimetry (DSC) was conducted on a TA Discovery 2500 with a T_{zero} pan and T_{zero} hermetic lid with a pin hole of 0.7 mm in diameter. DSC analysis was performed by ramping 10° C./min or 20° C./min from 30 to 250° C. or 0 to 250° C.

Thermal gravimetric analysis (TGA) was conducted on Discovery 5500 or Q5000 with a start temperature at ambient conditions (below 35° C.) and a final temperature of 300° C. (or abort next segment if weight <80% (w/w)) with a heat 10° C./min.

Nuclear magnetic resonance (NMR) analysis was run on a Bruker Avance-AV 400M at a frequency of 400 MHz for eight scans.

Dynamic vapor sorption (DVS) was performed on an Intrinsic, Advantage or Adventure with an oven temperature of 25° C. using water as a solvent. The sample mass was about 5-10 mg using the following method:
Cycle: 40-0-95-0-40% RH
Stage Step: 10%
Equilibrium: 0.002 dm/dt (%/min)
Minimum dm/dt stability duration: 60 min
Maximum dm/dt stage time: 360 min.

### Analysis of Polymorphs

3-(5-(2-hydroxy-2-methylpropoxy)-6-methylpyrazin-2-yl)-1H-indole-7-carbonitrile free form shows complicated polymorphic behaviors. In total, twenty-five (25) forms were identified to be polymorphs or pseudo-polymorphs of the free form, including eleven (11) anhydrates, Forms A, C, H, J, K, L, O, P, R, X, and Y; five (5) hydrates, Forms B, F, N, S, and T; and five (5) solvates, Forms D, E, I, Q, and U. In addition, four (4) metastable forms G, M, V, and W; and one (1) amorphous form were also obtained.

Form A is an anhydrate. It was obtained from many solvent systems by equilibration, slow cooling, fast cooling, evaporation, vapor diffusion and antisolvent addition experiments. Form A is of high crystallinity. DSC shows a melting point peak at Tₒₙₛₑₜ of 170.2° C. and then follows another endothermic peak at Tₒₙₛₑₜ of 178.9° C. TGA shows about 0.2% weight loss at about 160° C. ¹H-NMR shows no detectable residual solvent. Form A converts to Form C at both 25° C. and 50° C., suggesting that Form A is thermodynamically less stable than Form C.

Form B is a hydrate. It was only obtained from methanol system by temperature cycle, slow cooling, slow evaporation and fast evaporation. Form B is of high crystallinity. DSC shows a dehydration peak at Tₒₙₛₑₜ at 16.7° C. with an enthalpy of about 22 J/g and a small exothermic peak at Tₒₙₛₑₜ of 130.5° C. Then 2 un-resolved endothermic peaks at Tₒₙₛₑₜ of 170.2° C. and Tₒₙₛₑₜ of 173.1° C., respectively. After that, it shows two small unresolved endothermic peaks at Tₒₙₛₑₜ of 177.8° C. and Tₒₙₛₑₜ of 179.0° C., respectively. TGA shows about 2.1% weight loss at about 150° C. Karl Fischer titration (KF) shows it contains about 3.8% water by weight, equivalent to 0.7 water molecule. ¹H-NMR shows no detectable residual solvent. Form B shows reversible dehydration-hydration behavior. It converts to Form K after dehydration and Form K reverts back to Form B after exposure to 25° C. in 92% RH for 6 days. Form B is a metastable hydrate and it converts to anhydrate Form C in the whole range of water activity (0≤a.w.≤1).

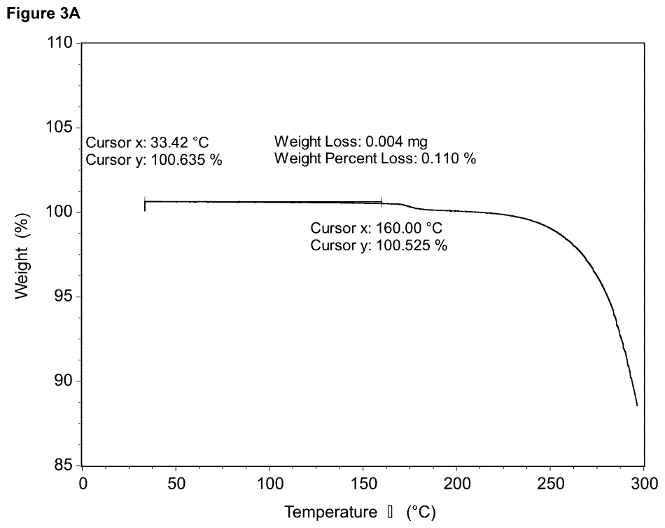

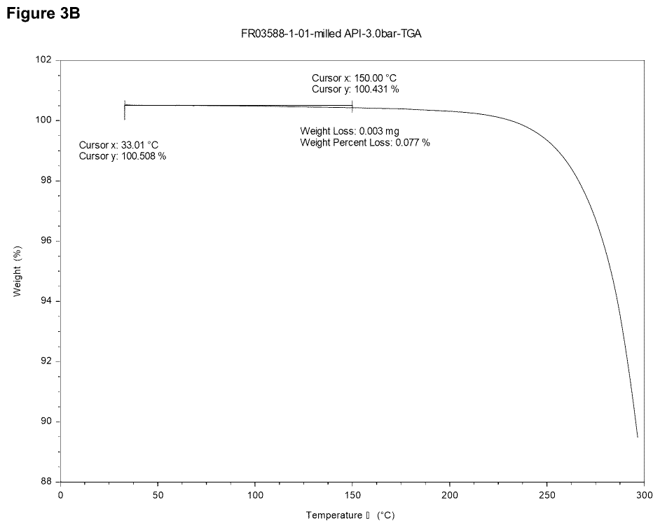

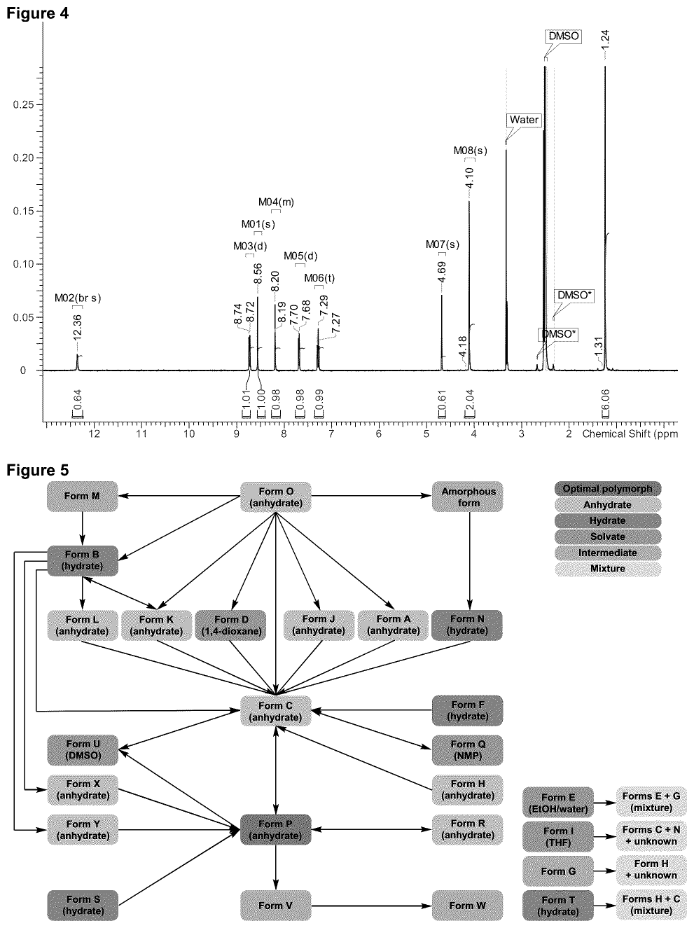

Form C is an anhydrate. It was obtained from some of solvent systems by equilibration, slow cooling, fast cooling, slow evaporation and vapor diffusion. Form C is of high crystallinity. DSC shows a melting peak at Tₒₙₛₑₜ of 179.3° C. with an enthalpy of about 114 J/g (FIG. 2). TGA shows about 0.8% weight loss at about 150° C. (FIG. 3). 1H-NMR shows no detectable residual solvent (FIG. 4). Water activity study shows that Form C is stable in the whole range of water activity (0≤a.w.≤1).

Form D is a 1, 4-dioxane solvate. It was only obtained from 1,4-dioxane system by equilibration, slow cooling, fast cooling, slow evaporation, fast evaporation, and vapor diffusion experiments. Form D is of high crystallinity. DSC shows two desolvation peaks at Tₒₙₛₑₜ of 88.0° C. with an enthalpy of about 27 J/g and at Tₒₙₛₑₜ of 138.6° C. with an enthalpy of about 41 J/g, suggesting that 1,4-dioxane molecules have two types of intermolecular interaction formats with the API molecule in crystal structure of Form D. Then it melts at Tₒₙₛₑₜ of 179.1° C. with an enthalpy of about 111 J/g. TGA shows about 9.1% weight loss at about 130° C. and about 2.8% weight loss from about 130° C. to 160° C. ¹H-NMR shows 0.5 equiv. (13.6%) 1, 4-dioxane residue by weight. Form D converts to Form C after desolvation.

Form E is an EtOH/water solvate. It was obtained from ethanol system by fast evaporation experiments. Form E is of high crystallinity. DSC shows a desolvation/dehydration peak at Tₒₙₛₑₜ of 80.4° C. with an enthalpy of about 8 J/g and un-resolved melting peaks at Tₒₙₛₑₜ of 165.2° C. and 170.2° C., respectively. This un-resolved melting peak is followed with a recrystallization peak at Tₒₙₛₑₜ of 173.1° C. After recrystallization, it shows a melting point at Tₒₙₛₑₜ of 177.9° C. that is close to the melting of Form C. TGA shows about 2.1% weight loss at about 120° C. ¹H-NMR shows 1.4% EtOH by weight. KF shows that it contains about 0.6% water by weight. Form E converts to physical mixture of Form F and Form G after heating to 100° C. by TGA and cooled to room temperature.

Form F is a hydrate. It was obtained from THF or MEK system by fast evaporation experiments. Form F is of high crystallinity. DSC shows 2 un-resolved melting peaks at Tₒₙₛₑₜ of 163.2° C. and 169.7° C. combined with a recrystallization peak at Tₒₙₛₑₜ of 171.7° C. After recrystallization, it shows a melting point at Tₒₙₛₑₜ of 177.9° C. that is close to the melting of Form C. TGA shows about 0.5% weight loss at about 140° C. and about 0.7% weight loss from about 140° C. to 180° C. 1H-NMR shows 0.5% THF residue by weight. KF shows it contains about 1.7% water by weight. Form F is a metastable hydrate and it converts to Form C in the whole range of water activity (0≤a.w.≤1).

Form G is a metastable form. It was obtained from DMSO/water system by anti-solvent addition experiments. Form G is of high crystallinity. It converts to anhydrate Form H after equilibration in DMSO/water system at 25° C. for 6 days.

Form H is an anhydrate. It was obtained from acetone or acetone/water (v:v=20:80) by slow cooling experiments. Form H is of high crystallinity. DSC shows un-resolved melting peaks at Tₒₙₛₑₜ of 169.3° C. and 173.2° C., respectively. After that, it shows a small endothermic peak at Tₒₙₛₑₜ of 179.2° C. TGA shows about 1.2% weight loss at about 150° C. ¹H-NMR shows no detectable residual solvent. Form H is a metastable anhydrate. It converts to Form C at both 25° C. and 50° C., suggesting that Form H is thermodynamically less stable than Form C.

Form I is a THF solvate. It was obtained from THF/heptane system by anti-solvent addition experiments. Form I is of high crystallinity. DSC shows a desolvation peak at Tₒₙₛₑₜ of 92.8° C. with an enthalpy of about 73 J/g. Then it melts at Tₒₙₛₑₜ of 178.5° C. with an enthalpy of about 114 J/g. TGA shows about 10.0% weight loss at about 130° C. ¹H-NMR shows 0.5 equiv. (10.1%) THF residue by weight. Form I converted to physical mixture of Form C, Form N and an unknown peak after heating to 140° C. by TGA and cooled to room temperature.

Form J is an anhydrate. It was obtained by heat-cool DSC. Form J is of high crystallinity. DSC shows a small exothermic event with an onset of 94.0° C., a melting point peak at Tₒₙₛₑₜ of 172.1° C. and then follows another endothermic peak at Tₒₙₛₑₜ of 177.4° C. TGA shows about 0.6% weight loss at about 150° C. ¹H-NMR shows no detectable residual solvent. Form J is a metastable anhydrate and it converts to Form C at both 25° C. and at 50° C., suggesting that Form J is thermodynamically less stable than Form C.

Form K is an anhydrate. It was obtained from methanol system from equilibration, fast cooling experiments and dehydration of Form B. Form K is of high crystallinity. DSC shows two unresolved melting peaks at Tₒₙₛₑₜ of 168.7° C. and 171.9° C. combined with a recrystallization peak at Tₒₙₛₑₜ of 173.4° C. After recrystallization, it shows two unresolved melting peaks at Tₒₙₛₑₜ of 177.3° C. and 178.8° C., respectively. Form K is unstable and it converts to hydrate Form B after exposure to 25° C./92% RH for 6 days.

Form L is an anhydrate. It was obtained after heated Form B to 160° C. and cooled to room temperature. Form L is of high crystallinity. Based on DSC thermogram and VT-XRPD results, Form K converts to Form L after melting and re-crystallization. Form L melts at Tₒₙₛₑₜ of 173.5° C. with an enthalpy of about 86 J/g. Form L is a metastable anhydrate, and it converts to Form C at both 25° C. and 50° C., suggesting that Form L is thermodynamically less stable than Form C.

Form M is an metastable crystalline form. It was obtained from methanol system by equilibration. Form M is of high crystallinity. It converts to Form B after equilibration in MeOH at 25° C. for another 1 day.

Form N is a hydrate. It was obtained from exposing amorphous form to 25° C./92% RH in an open container for 3 days. Form N is of high crystallinity. DSC shows a dehydration peak at Tₒₙₛₑₜ of 47.1° C., an exothermic peak at Tₒₙₛₑₜ of 81.1° C. and an endothermic peak at Tₒₙₛₑₜ of 128.5° C. with an enthalpy of about 11 J/g. Then it melts at Tₒₙₛₑₜ of 171.6° C. with an enthalpy of about 76 J/g. Two small endothermic peaks with Tₒₙₛₑₜ of 177.3° C. and 178.8° C. are shown after melting. KF shows it contains about 8.7% water by weight, equivalent to 1.5 water molecules. Form N is a metastable hydrate and it converts to Form C in the whole range of water activity (0≤a.w.≤1).

Form O is an anhydrate. It was obtained from temperature cycle, slow evaporation and fast evaporation in some solvent systems. Form O is of high crystallinity. DSC shows unresolved melting peaks at Tₒₙₛₑₜ of 170.1° C. and 178.5° C., respectively. TGA shows about 0.7% weight loss at about 180° C. 1H-NMR shows no detectable residual solvent.

Form P is an anhydrate. It was obtained from equilibration in pH 1.2 HCl buffer or temperature cycle in acetone. Form P is of high crystallinity. DSC shows a melting peak of Form P at Tₒₙₛₑₜ of 169.5° C., a recrystallization peak at 172.2° C., a small endothermic peak at 173.5° C. (which could be melting point of Form L), a recrystallization peak at 174.0° C. and a melting peak of Form C at Tₒₙₛₑₜ of 179.4° C. with an enthalpy of about 87 J/g. TGA shows about 0.1% weight loss at about 160° C. ¹H-NMR shows no detectable residual solvent. Form P is the most stable polymorph identified between 25° C. and 70° C. so far. Competitive equilibration experiments show that Form P is thermodynamically more stable than the other anhydrates between 25° C. and 80° C., while Form C could be thermodynamically more stable than Form P above 90° C. The XRPD of Form P is shown in FIG. 1A.

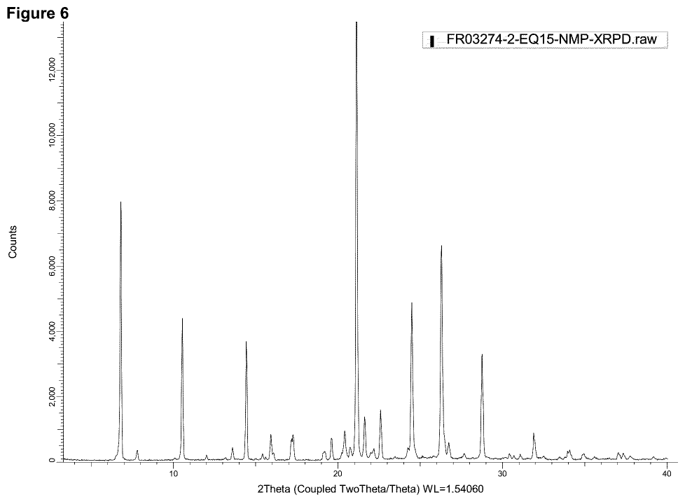

Form Q is a N-methyl-2-pyrrolidone (NMP) solvate. It was obtained from equilibration in NMP. Form Q is of high crystallinity. DSC shows desolvation peaks from about 78.7° C. and 86.9° C. and a broad endothermic peak form about 152° C. TGA shows about 22.4% weight loss at about 150° C. and about 2.3% weight loss from 150° C. to 200° C. 1H-NMR shows 1.0 equiv. of NMP by molar ration (about 23.5% by weight). Form Q converted to a physical mixture of Form C (majority) and Form Q after desolvation by heating to 150° C. The XRPD of Form Q is shown in FIG. 6.

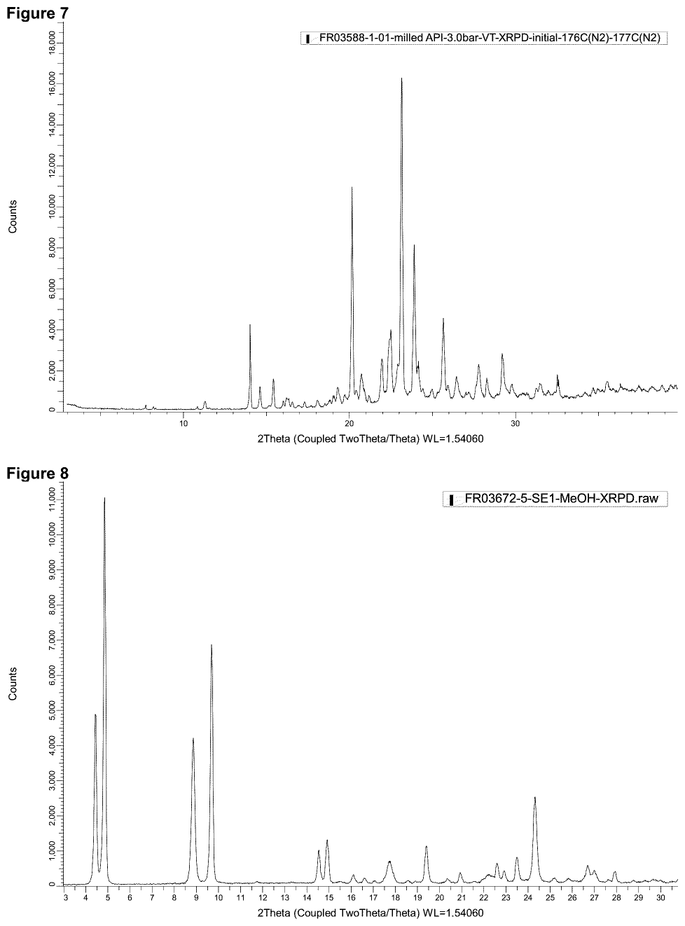

Form R is an anhydrate. It was obtained by heating milled Form P (batch FR03588-1-01-milled API-3.0bar) from 176° C. to 180° C. Form R is of high crystallinity. Competitive equilibration experiments show that Form R converts to Form P at both 25° C. and 70° C., suggesting that Form R is thermodynamically less stable than Form P. The XRPD of Form R is shown in FIG. 7.

Form S is a hydrate. It was obtained from slow evaporation in MeOH. Form S is of high crystallinity. DSC shows a dehydration peak from about 30° C., an endothermic peak at Tₒₙₛₑₜ of 124.8° C., a melting peak at Tₒₙₛₑₜ of 173.2° C. and a small endothermic peak at Tₒₙₛₑₜ of 178.0° C. TGA shows about 2.9% weight loss at about 170° C. 1H-NMR shows no detectable residual solvent. KF shows it contains 5.4% water by weight, equivalent to 1.0 water molecule. Form S is a metastable form. It converted to anhydrate Form P in the whole range of water activity (0≤a.w.≤1). The XRPD of Form S is shown in FIG. 8.

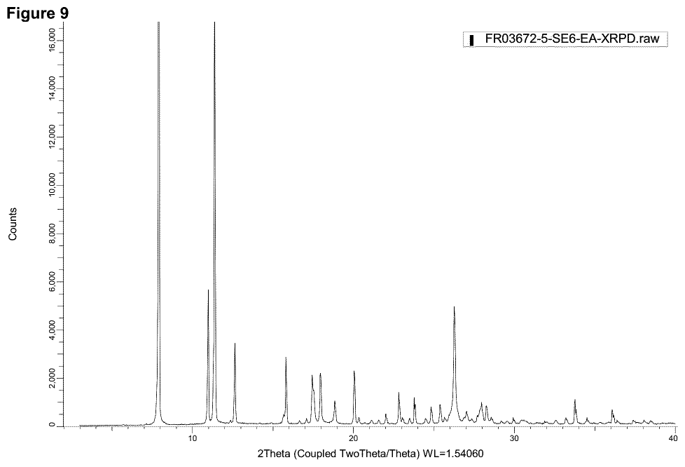

Form T is hydrate. It was obtained from slow evaporation in EA. Form T is of high crystallinity. DSC shows a dehydration peak form about 69° C., an endothermic peak at Tₒₙₛₑₜ of 170.0° C., an exothermic peak at Tₒₙₛₑₜ of 173.0° C. and a melting peak at Tₒₙₛₑₜ of 179.4° C. with an enthalpy of about 115 J/g. TGA shows about 3.1% weight loss at about 170° C. 1H-NMR shows 0.3% EA residue by weight (0.01 equiv. by molar ratio). Form T was difficult to be re-prepared and should be a metastable form. A physical mixture of Form H and Form C was obtained during re-preparation of Form T with the addition of seeds. The XRPD of Form T is shown in FIG. 9.

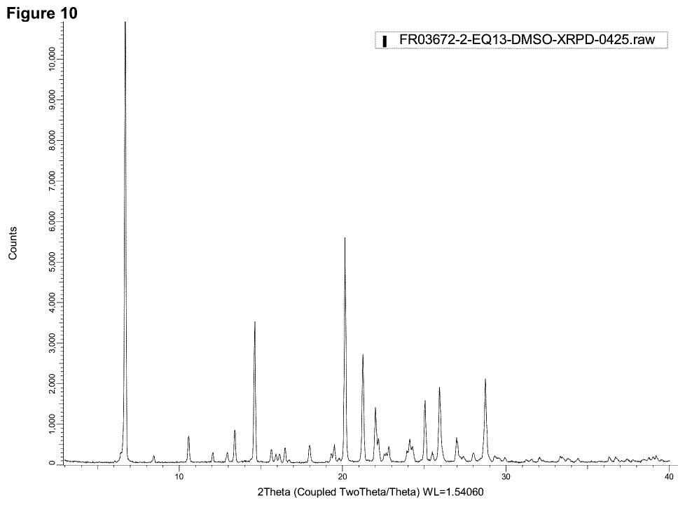

Form U is a DMSO solvate. It was obtained from equilibration in DMSO. Form U is of high crystallinity. DSC shows desolvation peaks from about 80.1° C. and 88.0° C. and a broad endothermic peak form about 151° C. TGA shows about 21.3% weight loss at about 150° C. and about 0.5% weight loss from 150° C. to 200° C. ¹H-NMR shows 1.1 equiv. of DMSO by molar ratio (about 23.1% by weight). Form U converted to Form C after desolvation by heating. The XRPD of Form U is shown in FIG. 10.

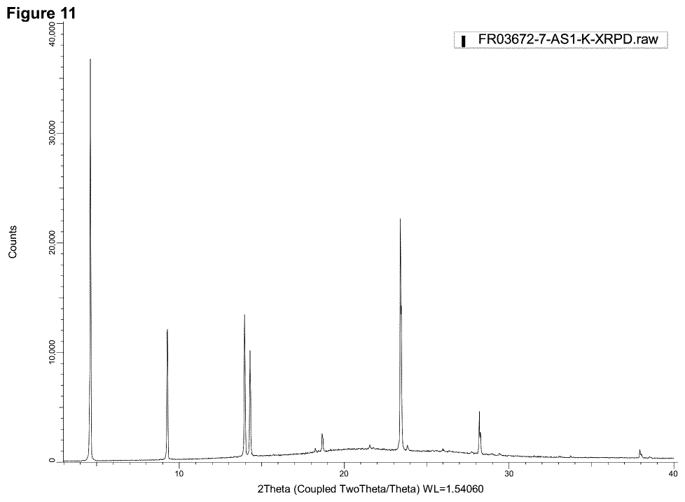

Form V is a metastable crystalline form. It was obtained from MeOH and captured under protection of Kapton film. Form V is of high crystallinity. Form V was unstable at ambient condition (20° C.-27° C., 50% RH-70% RH) and converted to Form W within 12 hours. The XRPD of Form V is shown in FIG. 11.

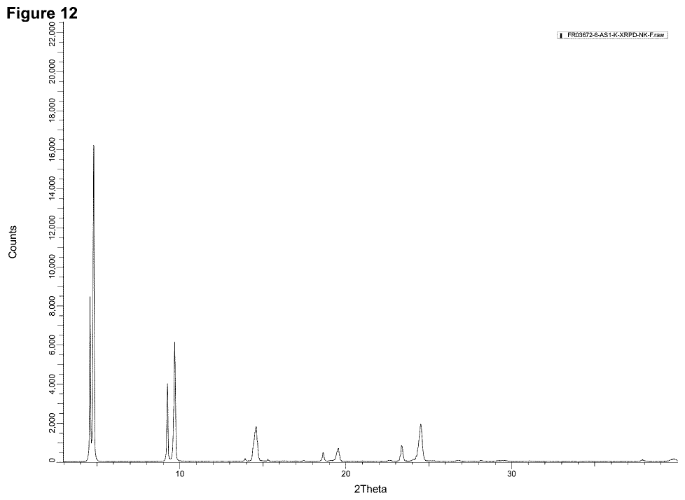

Form W is a metastable crystalline form. It was obtained by exposure Form V at ambient condition (20° C.-27° C., 50% RH-70% RH). Form W is of high crystallinity. Form W was non-reproducible and should be a metastable form. Form B was obtained with addition of Form W seeds. The XRPD of Form W is shown in FIG. 12.

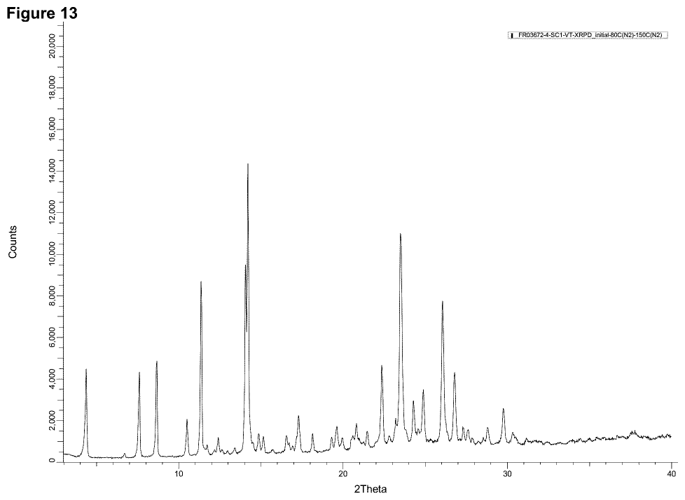

Form X is an anhydrate. It was obtained by heating Form B to 150° C. Form X is of high crystallinity. Competitive equilibration experiments show that Form X converts to Form P at 25° C., 50° C. and 70° C., suggesting that Form X is thermodynamically less stable than Form P between 25° C. and 70° C. The XRPD of Form X is shown in FIG. 13.

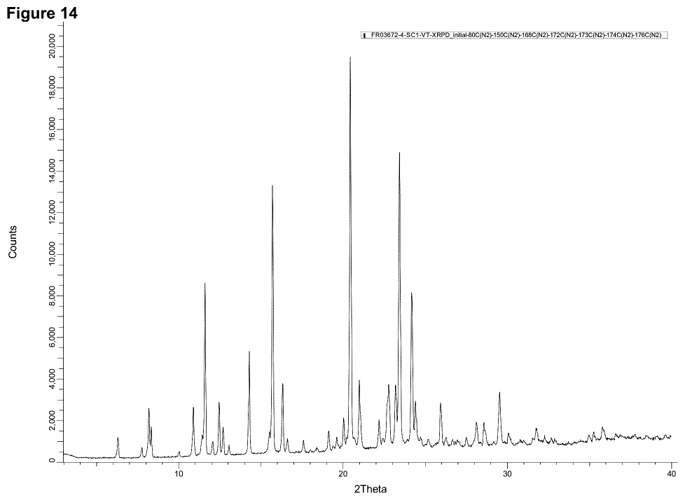

Form Y is an anhydrate. It was obtained by heating Form B to 176° C. Form Y is of high crystallinity. Competitive equilibration experiments show that Form Y converts to Form P at 25° C., 50° C. and 70° C., suggesting that Form Y is thermodynamically less stable than Form P between 25° C. and 70° C. The XRPD of Form Y is shown in FIG. 14.

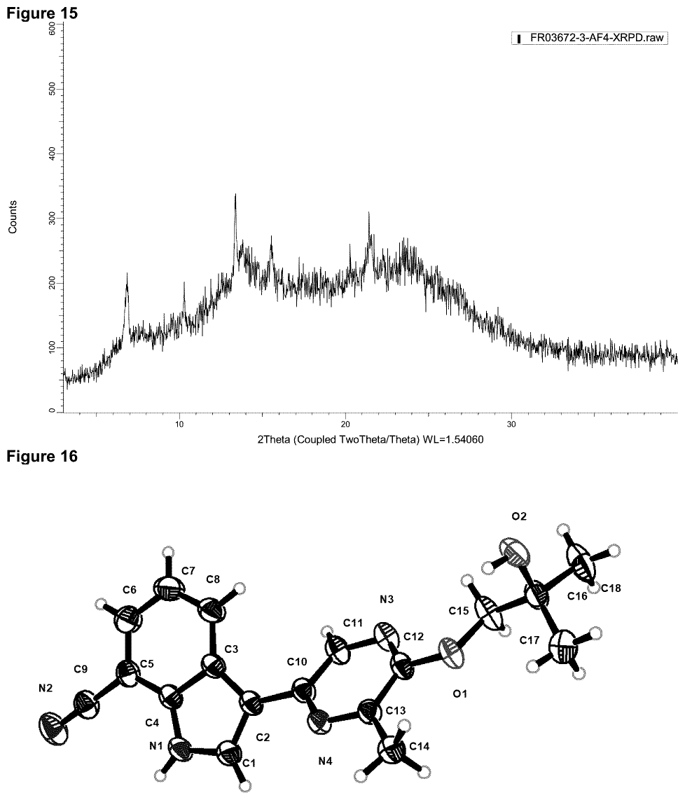

An amorphous form was prepared by heat-cool TGA using the free form. The amorphous form crystallized to Form N after exposure to high humidity. The XRPD of the amorphous form is shown in FIG. 15.

Table 2 outlines the screening conditions of the polymorphs discussed above.

TABLE 2
Summary of screening conditions of polymorphs

| Polymorphs | Screening experiments |
|---|---|
| Form A, anhydrate | From most of solvent systems by equilibration, slow cooling, fast cooling, slow |
| | evaporation, fast evaporation, vapor diffusion and antisolvent addition |
| | experiments |
| Form B, hydrate | From methanol system by temperature cycle, slow cooling, slow evaporation |
| | and fast evaporation |
| Form C, anhydrate | From most of solvent systems by equilibration, slow cooling, fast cooling, slow |
| | evaporation and vapor diffusion. |
| Form D, solvate | From 1,4-dioxane system by equilibration, slow cooling, fast cooling, slow |
| | evaporation, fast evaporation, and vapor diffusion experiments. |
| Form E, solvate | From ethanol system by fast evaporation experiments |
| Form F, hydrate | From THE or MEK system by fast evaporation experiments |
| Form G | From DMSO/Water system by antisolvent experiments |
| Form H, anhydrate | From acetone or acetone/water (v:v = 20:80) by slow cooling experiments |
| Form I, solvate | From THF/Heptane system by antisolvent experiments for 6 days |
| Form J, anhydrate | By heat-cool DSC |
| Form K, anhydrate | From methanol system from equilibration, fast cooling experiments and Form |
| | B after dehydration |
| Form L, anhydrate | After heated Form B to 160° C. and cooled to room temperature |
| Form M | From methanol system by equilibration |
| Form N, hydrate | Exposure amorphous form to 25° C./92% RH in an open container for 3 days |
| Form O | From water system by temperature cycle, IPAc system by slow evaporation, |
| | acetonitrile (MeCN) system by fast evaporation |
| Form P, anhydrate | From pH 1.2 HCl buffer by equilibration, from acetone by temperature cycle |
| Form Q, NMP | From NMP by equilibration |
| solvate | |
| Form R, anhydrate | After heated milled Form P from 176° C. to 180° C. |
| Form S, hydrate | From MeOH by slow evaporation |
| Form T, hydrate | From EA by slow evaporation |
| Form U, DMSO | From DMSO by equilibration |
| solvate | |
| Form V | From MeOH covered with Kapton film |
| | Exposure Form V at ambient condition (20-27° C., 50% RH-70% RH) |
| Form W | |
| Form X, anhydrate | After heated Form B to 150° C. by VT-XRPD |
| Form Y, anhydrate | After heated Form B to 176° C. by VT-XRPD |
| Amorphous form | Prepared by heat-cool TGA using 3-(5-(2-hydroxy-2-methylpropoxy)-6- |
| | methylpyrazin-2-yl)-1H-indole-7-carbonitrile free form |

### Preparation of Form P

Form P was prepared using the following procedure:

3-(5-(2-hydroxy-2-methylpropoxy)-6-methylpyrazin-2-yl)-1H-indole-7-carbonitrile (˜5 g, free form) was weighed into a 150 mL glass beaker. 30 mL of 2-MeTHF was added into the glass beaker at 50° C. with stirring at a rate of 200 rpm to obtain a clear solution. The clear solution was cooled to about 25° C. at a rate of 0.1° C./min. A hazy suspension was obtained. About 50 mg of Form P seeds was added into above hazy suspension. Then the hazy suspension was cooled to about 5° C. at a rate of 0.1° C./min. The hazy suspension became a suspension. The suspension was stirred at 5° C. for about 12 hours. 20 mL of heptane was added into above suspension and stirring continued at 5° C. for about 12 hours. Solids were collected by centrifugation and then dried at 25° C. under vacuum for about 12 hours. 3.6 g of Form P was obtained as an off-white solid in 72% yield.

### Alternative Preparations of Form P

Form P was prepared using the following alternative procedures:

Form C (˜9.5 g, chemical purity 100%) was added into 25 mL of acetone. The mixture was stirred at 50° C. After stirring at 50° C. for about 5 min, about 240 mg of Form P seeds were added into above suspension. Then the suspension was equilibrated under a temperature cycle between 5° C. to 50° C. at a heating/cooling rate of 0.1° C./min. After about 3 cycles, a few solids were collected by centrifugation and Form P was obtained. About 40 g of Form C (chemical purity 100%) and 65 mL of acetone was added into above suspension. After about 7 cycles, solids were collected by suction filtration and then dried at 25° C. under vacuum for about 1 day. 49 g of the Form P was obtained as an off-white solid in 98% yield.

Form C (0.3 g, chemical purity 100%) was added into 10 mL saturated solution of pH 1.2 HCl buffer. The mixture was stirred at 37° C. After stirring at 37° C. for about 24 h, a few solids were collected by centrifugation. The Form P was obtained. Form C (1.7 g) and 13 mL of pH 1.2 HCl buffer were added into above solution. The mixture was still stirred at 37° C. for about 2 days. Solids were collected by centrifugation and then dried at 37° C. under vacuum for about 2 hours. Form P (1.8 g) was obtained as an off-white solid in 90% yield.

Form C (60 mg, chemical purity 100%) was added into 0.2 mL of acetone. The mixture was stirred at 50° C. After stirring at 50° C. for about 5 mins, Form P seeds (50 mg) were added into above suspension. Then the suspension was equilibrated under a temperature cycle between 5° C. to 50° C. at a heating/cooling rate of 0.1° C./min for about 20 cycles. Solids were collected by centrifugation. Form P was obtained.

### Interrelationship of Polymorphs

The interconversion relation of polymorphs identified is outlined in FIG. 5.

Relative stability of the anhydrate Forms A, C, H and J was investigated with competitive equilibration experiments. Form C was obtained in all selected solvents at both 25° C. and 50° C. except in MeOH, where Form B was obtained. Intermediate crystalline form, Form M, was obtained from methanol as a wet cake and further converted to Form B readily once exposed to the air. The presence of intermediate Form M affects the competitive equilibration experiments and cannot reflect the real thermodynamic relationships among anhydrates in methanol. In other solvent systems selected, Form C is the most stable form.

Relative stability of the anhydrates Forms C, K and L was investigated with competitive equilibration experiments. Form C was obtained in IPAc both at 25° C. and at 50° C.

Relative stability of anhydrate Form C, hydrate Form B, and hydrate Form F was investigated by competitive water activity experiments. Relative stability of the anhydrate Form C and the hydrate Form N was also investigated by water activity experiments in acetone/water solvent mixture (a.w.=0.95). Results show that Form C is the thermodynamically stable form in the whole range of water activity (0≤a.w.≤1).

Form P was first obtained in solubility study of Form C at 37° C. This indicated that Form P is thermodynamically more stable than Form C at 37° C. Since Form P shows a lower melting point than that of the Form C, Form C and Form P are likely enantiotropically related with Form P being the more stable form at low temperature and Form C the more stable form at high temperature. Relative stability of the anhydrates Form C and Form P was systematically investigated with competitive equilibration experiments. The solvents used in these experiments are listed in Table 3. Form P was obtained at 25° C., 50° C., 70° C. and 80° C., suggesting that Form P is thermodynamically more stable than Form C at 80° C. and below. At 90° C., Form P converted to Form C in toluene and in DMSO/water, while Form C converted to Form P in water. This suggests that transition temperature between Form C and Form P is likely close to 90° C.

Relative stability of the anhydrates Form R and Form P was investigated by variable temperature XRPD and competitive equilibration experiments. Form P converted to Form R when heated above 176° C. using milled Form P. Form R converted to Form P between 25° C. and 70° C. in 2-MeTHF by competitive equilibration, indicating that Form P is more stable than Form R in the temperature range of 25° C. to 70° C.

Relative stability of the anhydrates Form X, Form Y and Form P was investigated with competitive equilibration experiments. Form P was obtained at 25° C., 50° C. and 70° C., suggesting that Form P is thermodynamically more stable than Form X and Form Y between 25° C. and 70° C.

Relative stability of anhydrate Form P and hydrate Form S was investigated by competitive water activity experiments. Results show that Form P is thermodynamically more stable than hydrate Form S in the whole range of water activity (0≤a.w.≤1) at 25° C.

Relative stability of the anhydrates Form B, Form X, Form Y and Form C was investigated by variable temperature XRPD. Form B converted to Form X, Form Y and Form C after heating to 150° C., 176° C. and 180° C., respectively.

To investigate form conversion kinetics from Form C to Form P, equilibration experiments with Form C in pH 1.2 HCl buffer, 2-MeTHF, heptane and water were conducted. In 2-MeTHF, Form C converted to Form P within 2 h at 25° C. In pH 1.2 HCl buffer, Form C converted to Form P after 31 h at 37° C. In heptane, Form C converted to Form P completely after 1 week at 25° C. In water, Form C converted to Form P completely after 4 days at 25° C.

### Competitive Equilibration Experiments

To determine relative stability of Form C and Form P, competitive equilibration experiments were conducted in different solvent systems.

About 5 mg of Form C and about 5 mg of Form P were added to 0.25 mL of saturated solutions of selected solvents. Obtained suspensions were stirred at 25° C., 50° C. and 70° C. for 4 days, respectively. Solid parts (wet cakes) were isolated by centrifugation filtration and investigated by XRPD. The results are shown in Table 3.

TABLE 3
Competitive equilibration experiments between Form C and Form P-1
Initial polymorphs: Form C and Form P

| | XRPD | | |
|---|---|---|---|
| | 25° C. for | 50° C. for | 70° C. for |
| Solvents | 4 days | 4 days | 4 days |
| Ethanol | Form P | Form P | Form P |
| Acetonitrile | Form P | Form P | Form P |
| Ethyl acetate | Form P | Form P | Form P |
| Isopropyl acetate | Form P | Form P | Form P |
| 1,4-Dioxane | Form D | Form D | Form D |
| MEK | Form P | Form P | Form P |
| Acetone | Form P | Form P | Form P |
| Acetone/water | Form P | Form P | Form P |
| (v:v = 95.2:4.8) | | | |
| a.w = 0.4 | | | |
| Acetone/water | Form P | Form P | Form P |
| (v:v = 76.5:23.5) | | | |
| a.w = 0.7 | | | |
| Acetone/water | Form P | Form P | Form P |
| (v:v = 36:64) a.w = 0.9 | | | |
| NMP | Form Q, NMP | // | // |
| | solvate; | | |
| | ¹H-NMR: 1 equiv. | | |
| | NMP | | |
| Water | Form P | Form P | Form P |
| Heptane | Form P | Form P | Form P |
| 2-MeTHF | Form P | Form P | Form P |
| DCM | Form P | // | // |
| THF | Form P | Form P | // |

// = No comments.

To determine relative stability of Form P and Form C at high temperature, competitive equilibration experiments were conducted in different solvent systems at 80° C. and 90° C.

About 10 mg of Form C and about 10 mg of Form P were added to 0.5 mL saturated solutions of selected solvents. Obtained suspensions were stirred at 80/C and 90 C, respectively. Solid parts (wet cakes) were be isolated by centrifugation filtration and investigated by XRPD. The results are shown in Table 4.

TABLE 4
Competitive equilibration experiments
between Form C and Form P-2

| | XRPD | | |
|---|---|---|---|
| | 80° C. for | 90° C. for | 90° C. for |
| Solvents | about 6 d | about 8 d | about 18 d |
| Toluene | // | Form C | // |
| Heptane | // | Form C + Form P | Form C + Form P |
| Water | // | Form C + Form P | Form P |
| DMSO/water | Form P | Form C (majority) + | Form C |
| (v:v = 1:5 | | Form P | |
| a.w. = 0.94) | | | |

// = No comments.

Water activity in Table 4 was calculated by UNIFAC method.

The following competitive equilibration experiments among Form P and other anhydrates were conducted.

To determine relative stability of the Form P and the mixture of Form P and Form J, competitive equilibration experiments were conducted in different solvent systems.

About 3 mg of the Form P and 3 mg of the mixture of Form P and Form J were added to 0.2 mL of saturated solutions of selected solvents. Obtained suspensions were stirred at 25° C., 50° C. and 70° C. for 4 days, respectively. Solid parts (wet cakes) were isolated by centrifugation filtration and investigated by XRPD. The results are shown in Table 5.

TABLE 5
Competitive equilibration experiments between Form P and Form J
Initial polymorphs: Form P and mixture of Form P and Form J

XRPD

| | 25° C. for | 50° C. for | 70° C. for |
|---|---|---|---|
| Solvents | 4 days | 4 days | 4 days |
| Acetone | Form P | Form P | Form P |
| 2-MeTHF | Form P | Form P | Form P |

To determine relative stability of the Form P and Form R, competitive equilibration experiments were conducted in 2-MeTHF.

About 3 mg of Form P and 3 mg of the physical mixture of Form P and Form R were added to 0.2 mL of saturated solutions of 2-MeTHF. Obtained suspensions were stirred at 25° C., 50° C. and 70° C., respectively. Solid parts (wet cakes) were isolated by centrifugation filtration and investigated by XRPD. The results are shown in Table 6.

TABLE 6
Competitive equilibration experiments between Form P and Form R
Initial polymorphs: Form P + physical mixture of Form P and Form R

| | XRPD | | | |
|---|---|---|---|---|
| | 25° C. for | 50° C. for | 70° C. for | 70° C. for |
| Solvents | 3 days | 3 days | 3 days | 1 week |
| 2-MeTHF | Form P | Form P | Form P + one peak | Form P |
| | | | of Form R | |

To determine relative stability of Form P, Form Y and Form X, competitive equilibration experiment was conducted in 2-MeTHF at 25° C., 50° C. and 70° C., respectively.

About 1.5 mg of Form P, 1.5 mg of Form X and 1.5 mg Form Y were added to 0.15 mL saturated solution of 2-MeTHF. Obtained suspensions were stirred at 25° C., 50° C. and 70° C., respectively. Solid parts (wet cake) were isolated by centrifugation filtration and investigated by XRPD. The results are shown in Table 7.

TABLE 7
Competitive equilibration experiment among Form P, Form X and Form Y
Samples: a physical mixture of Form P + Form X + Form Y

| | 25° C. for | 50° C. for | 70° C. for |
|---|---|---|---|
| Solvent | about 3 days | about 3 days | about 3 days |
| 2- | Form P | Form P | Form P |
| MeTHF | | | |

Form P was confirmed to be thermodynamically more stable than the other anhydrates between 25° C. and 70° C. in competitive equilibration experiments. Additionally, in a competition slurry with Form C at 80° C. in DMSO/water, only Form P remained. Thus, it is likely that the transition temperature between Form P and Form C is in the range of 80-90° C. Therefore, Form C and Form P are likely enantiotropically related with Form P being the more stable form at low temperature and Form C the more stable form at high temperature.

### Physicochemical Characteristics

The physicochemical characteristics of two separate preparations of Form P are summarized in Table 8.

TABLE 8
Physicochemical characteristics of Form P

| Polymorph | Form P | | |
|---|---|---|---|
| Parameters | | Results | |
| Preparation | Method | First | Second |
| Purity | HPLC | 100% | 100% |
| X-ray diffraction | 3-40° (2 | High crystallinity, | High crystallinity, Form P - shown |
| | theta) | Form P | in FIG. 1 |
| Thermal events and | DSC, | Melting onset | Melting onset 170.5° C., enthalpy |
| enthalpy | 10° C./min | 169.5° C., enthalpy | 112 J/g, a small endothermic peak at |
| | | 65 J/g; | 179.7° C. - shown in FIG. 2A |
| | | A small exothermic | |
| | | peak at 172.2° C.; | |
| | | A small endothermic | |
| | | peak at 173.5° C. | |
| | | A small exothermic | |
| | | peak at 174.0° C.; | |
| | | Melting onset | |
| | | 179.4° C., enthalpy | |
| | | 87 J/g | |
| Thermogravimetry | TGA, | 0.1% @ 160° C. | 0.1% @ 160° C. |
| | 10° C./min | | |
| Residual Solvent | ¹H-NMR | Undetected | Undetected |
| | (DMSO- | | |
| | d₆) | | |
| Morphology | SEM | Block-like, ranging | |
| | | from about 1 μm to | |
| | | 15 μm | |
| Morphology | PLM | Block-like, ranging | Block-like; ranging from about 5 μm |
| | | from about 1 μm to | to 10 μm |
| | | 15 μm | |
| Particle size | PSD | | // |
| distribution | | | |

// = No comments.

### Bulk Stability

Bulk stability of Form P was evaluated at 25° C./92% RH in an open container, at 40° C./75% RH in an open container and at 60° C. in a tight container over 1 week, 2 weeks and 4 weeks. Form P is physically and chemically stable after stressing under these 3 conditions over 1 week, 2 weeks and 4 weeks. Samples after the stress were characterized by XRPD and HPLC and inspected for color change. Form P was also stressed under visible light for 1.2 million lux-hrs. at 25° C. Samples after the stress were characterized by XRPD and HPLC and inspected for color change. This data is summarized in Table 9.

TABLE 9
Bulk stability of Form P

| | | 100% | |
|---|---|---|---|
| Exp. ID | Initial purity | Purity | Color |
| 1 | Solid state, 25° C./92.5% RH, open container, 1 week | | |
| | Bulk (HPLC) | 100% | No color change |
| | Bulk (XRPD) | No form change | |
| 2 | Solid state, 40° C./75% RH, open container, 1 week | | |
| | Bulk (HPLC) | 100% | No color change |
| | Bulk (XRPD) | No form change | |
| 3 | Solid state, 60° C., tight container, 1 week | | |
| | Bulk (HPLC) | 99.9% | No color change |
| | Bulk (XRPD) | No form change | |
| 4 | Solid state, photo (visible light, closed, 1.2 million lux-hrs at 25° C.) | | |
| | Bulk, clear vial (HPLC) | ~60% | medium discoloration |
| | Bulk, wrapped with | 100% | No color change |
| | aluminum foil (HPLC) | | |
| | Bulk, clear vial (XRPD) | No form change | |
| 5 | Solid state, 25° C./92.5% RH, open container, 2 weeks | | |
| | Bulk (HPLC) | 100% | No color change |
| | Bulk (XRPD) | No form change | |
| 6 | Solid state, 40° C./75% RH, open container, 2 weeks | | |
| | Bulk (HPLC) | 100% | No color change |
| | Bulk (XRPD) | No form change | |
| 7 | Solid state, 60° C., tight container, 2 weeks | | |
| | Bulk (HPLC) | 100% | No color change |
| | Bulk (XRPD) | No form change | |
| 8 | Solid state, 25° C./92.5% RH, open container, 4 weeks | | |
| | Bulk (HPLC) | 100% | No color change |
| | Bulk (XRPD) | No form change | |
| 9 | Solid state, 40° C./75% RH, open container, 4 weeks | | |
| | Bulk (HPLC) | 100% | No color change |
| | Bulk (XRPD) | No form change | |
| 10 | Solid state, 60° C., tight container, 4 weeks | | |
| | Bulk (HPLC) | 100% | No color change |

| | |
|---|---|
| Bulk (XRPD) | No form change |

### Solubility

Non-milled Form P (10 mg, purity 100%) was weighed into a 20 mL glass vial. 5 mL of solubility medium was added. Obtained suspensions were stirred at 37° C. at 400 rpm and sampled at 24 hours. The samples were centrifuged at 37 C at 14,000 rpm for 5 min. Supernatants were analyzed by HPLC and pH meter for solubility and pH value, respectively. Residual solids (wet cakes) from the 24 hours samples were also characterized by XRPD to determine physical form. Experimental results are summarized in Table 10.

TABLE 10
Solubility of non-milled Form P

| | Solubility at 37° C., target concentration 2 mg/mL, equilibration for 24 hours, LOQ: 0.5 μg/mL | | |
|---|---|---|---|
| Method | Solubility | Solubility | |
| Exp. ID | media | (μg/mL) (pH) | XRPD |
| 1 | pH 1.2 HCl buffer (0.1N) | 7.2 (1.1) | Form P |
| 2 | pH 3 citrate buffer (50 mM) | 2.9 (3.0) | Form P |
| 3 | pH 5 acetate buffer | 2.3 (4.9) | Form P + one additional peak (It |
| | (50 mM) | | may belong to sodium acetate) |
| 4 | pH 7 phosphate buffer | 2.4 (6.9) | Form P |
| | (50 mM) | | |
| 5 | FaSSGF, pH 1.6 | 5.1 (1.5) | Form P |
| 6 | FaSSIF-v1, pH 6.5 | 20.9 (6.4) | Form P |
| 7 | FeSSIF-v1, pH 5.0 | 110.7 (4.9) | Form P |
| 8 | Water | 2.7 (7.7) | Form P |

Form P (5 mg, purity 100%) was weighed to a 2 mL glass vial. 20 μL aliquots of each solvent were added to dissolve the drug substance at 25 C. About 10 mg of Form P was weighed to a 2 mL glass vial. 20 μL aliquots of each solvent were added to dissolve the drug substance at 50° C. Vortex and sonication were applied to assist dissolution. Max. volume of each solvent added is 1 mL. For obtained suspensions (Exp. ID 1, 10 and 11) after addition of 1 mL solvent at 25° C., they were placed at 50° C. Approximate solubility was determined by visual observation. The results of additional solubility studies are shown in Table 11.

TABLE 11
Approximate solubility at 25° C. and at 50° C.

Solubility (mg/mL)

| Exp. ID | Solvents | 25° C. | 50° C. |
|---|---|---|---|
| 1 | Water | <5 | <5 |
| 2 | Methanol | 31-42 | 33-50 |
| 3 | Ethanol | 19-23 | 33-50 |
| 4 | 2-Propanol | 8-10 | 14-17 |
| 5 | Acetone | 50-63 | 100-125 |
| 6 | Acetonitrile | 7-8 | 14-16 |
| 7 | THF | 125-250 | 250-500 |
| 8 | Ethyl acetate | 17-25 | 33-50 |
| 9 | MTBE | <5 | <10 |
| 10 | Toluene | <5 | <5 |
| 11 | Heptane | <5 | <5 |
| 12 | DCM | 5-6 | |
| 13 | Isopropyl acetate | 13-17 | 25-33 |
| 14 | 1,4-Dioxane | 125-250 | 250-500 |
| 15 | DMSO | >250 | >500 |
| 16 | MEK | 42-50 | 83-100 |
| 17 | 2-MeTHF | 83-125 | 100-125 |
| 18 | DMF | >250 | >500 |
| 19 | NMP | >250 | >500 |

// = No comments.

Equilibration with Solvents

Based on approximate solubility results, about 50 mg of Form P was equilibrated in 0.2-1 mL of solvents at 25° C. for 2 weeks with a stirring bar on a magnetic stirring plate at a rate of 300-400 rpm. Obtained suspensions were filtered through a 0.45 μm nylon membrane filter by centrifugation at 14,000 rpm. Solid parts (wet cakes) were investigated by XRPD. For samples with different XRPD patterns, additional analysis including DSC, TGA, ¹H-NMR were performed. The results of these experiments are summarized in Table 12.

TABLE 12
Equilibration of Form P with solvents at 25° C. for 2 weeks

| Solvents | XRPD | Additional analysis |
|---|---|---|
| Water | Form P | // |
| Methanol | Form B + Form W | // |
| Ethanol | Form P | // |
| 2-Propanol | Form P | // |
| Acetone | Form P | // |
| Acetonitrile | Form P | // |
| THF | Form P | // |
| Ethyl acetate | Form P | // |
| MTBE | Form P | // |
| Toluene | Form P | // |
| Heptane | Form P | // |
| Isopropyl acetate | Form P | // |
| DMSO | Form U | Dried under N₂, no form change. |
| | | DSC: Desolvation from about 80.1° C. and 88.0° C., |
| | | Broad endothermic peak from 151° C. |
| | | TGA: 21.3% @ 150° C., 0.5% @ 150° C.-200° C. |
| | | ¹H-NMR: 23.1% DMSO by weight (1.1 equivalent by molar ratio) |
| MEK | Form P | // |
| NMP | Form Q | ¹H-NMR: 23.5% NMP by weight (1.1 equivalent by molar ratio) |
| | | DSC: desolvation from about 78.7° C. and 86.9° C., |
| | | Broad endothermic peak from about 152° C. |
| | | TGA: 22.4% @ 150° C., 2.3% @ 150° C.-200° C. |
| 2-MeTHF/heptane (v:v = 1:1) | Form P | // |
| Ethanol/water (v:v = 40:60) | Form P | // |
| a.w. = 0.91 | | |
| Acetone/water (v:v = 30:70) | // | // |
| a.w. = 0.92 | | |
| THF/water (v:v = 80:20) | // | // |
| a.w. = 0.91 | | |
| DMSO/water (v:v = 40:60) | // | // |
| a.w. = 0.77 | | |
| 2-MeTHF | Form P | // |
| 2-MeTHF/heptane (v:v = 3:1) | Form P | |

// = No comments.

Based on approximate solubility results, about 30 mg of Amorphous form was equilibrated in 0.2-1 mL of solvents at 25° C. with a stirring bar on a magnetic stirring plate at a rate of 300-400 rpm. Obtained suspensions were filtered through a 0.45 μm nylon membrane filter by centrifugation at 14,000 rpm. Solid parts (wet cakes) were investigated by XRPD as shown in Table 13.

TABLE 13
Equilibration of almost amorphous form with solvents at 25° C.

| Solvents | XRPD |
|---|---|
| Water | Form P |
| Methanol | Form B |
| Ethanol | Form P |
| 2-Propanol | Form P |
| Acetone | Form P |
| Acetonitrile | Form P |
| THF | Form I |
| Ethyl acetate | Form P |
| MTBE | Form P |
| Toluene | Form C |
| Heptane | Form C |
| Isopropyl acetate | Form P |
| DMSO | // |
| MEK | Form P |
| NMP | // |
| 2-MeTHF/heptane (v:v = 1:1) | Form P |
| Ethanol/water (v:v = 40:60) | Form P |
| a.w. = 0.91 | |
| Acetone/water (v:v = 30:70) | Form P |
| a.w. = 0.92 | |
| THF/water (v:v = 80:20) | // |
| a.w. = 0.91 | |
| DMSO/water (v:v = 40:60) | Form P |
| a.w. = 0.77 | |
| pH 1.2 HCl/KCl buffer | Form P |
| 2-MeTHF | Form P |
| 2-MeTHF/heptane (v:v = 3:1) | Form P |

// = No comments.

Based on approximate solubility results, about 50 mg of Form P was equilibrated in 0.1-0.5 mL of solvents at 50° C. for 1 week with a stirring bar on a magnetic stirring plate at a rate of 300-400 rpm. Obtained suspensions were filtered through a 0.45 μm nylon membrane filter by centrifugation at 14,000 rpm. Solid parts (wet cakes) were investigated by XRPD as shown in Table 14.

TABLE 14
Equilibration of Form P with solvents at 50° C. for 1 week

| Solvents | XRPD | Comments |
|---|---|---|
| Water | Form P | // |
| Methanol | Form P | // |
| Ethanol | Form P | // |
| 2-Propanol | Form P | // |
| Acetone | Form P | // |
| Acetonitrile | Form P | |
| THF | // | Clear solution |
| | | (250 mg/mL) |
| Ethyl acetate | Form P | // |
| MTBE | Form P | // |
| Toluene | Form P | // |
| Heptane | Form P | // |
| Isopropyl acetate | Form P | |
| DMSO | // | Clear solution |
| | | (500 mg/mL) |
| MEK | Form P | // |
| NMP | | Clear solution |
| | | (500 mg/mL) |
| 2-MeTHF/heptane (v:v = 1:1) | Form P | // |
| Ethanol/water (v:v = 40:60) | Form P | // |
| a.w. = 0.91 | | |
| Acetone/water (v:v = 30:70) | Form P | // |
| a.w. = 0.92 | | |
| THF/water (v:v = 80:20) | | Clear solution |
| a.w. = 0.91 | | (500 mg/mL) |
| DMSO/water (v:v = 40:60) | Form P | // |
| a.w. = 0.77 | | |
| 2-MeTHF | Form P | // |
| 2-MeTHF/heptane (v:v = 3:1) | Form P | // |

// = No comments.

### Anti-Solvent Crystallization

Based on approximate solubility results, about 40 mg of Amorphous form was dissolved in the minimal amount of selected good solvents at ambient temperature (about 20-27° C.). 1-6 folds of anti-solvent were added into the obtained clear solutions slowly until a large amount of solids precipitated out. Precipitates were collected by centrifugation filtration through a 0.45 μm nylon membrane filter at 14,000 rpm. Solid parts (wet cakes) were investigated by XRPD as shown in Table 15.

TABLE 15
Crystallization by addition of anti-solvent

| Solvents | Anti-solvent | | XRPD after | |
|---|---|---|---|---|
| (mL) | (mL) | XRPD | 8 days | Comments |
| Methanol | Water (5.5) | Form C | Form P | Form V was captured when |
| (1.4) | | | | covered with Kapton film during |
| | | | | preparation of clear solution. |
| | | | | Form V converted to Form W |
| | | | | within 12 hours at ambient |
| | | | | temperature (20° C.-27° C.). |
| Ethanol | MTBE (12.0) | // | // | Clear solution |
| (2.0) | | | | |
| 2-Propanol | Heptane (15.0) | // | // | Clear solution |
| (5.0) | | | | |
| Acetone | Water (2.0) | Form C + | Form P | // |
| (1.0) | | Form G | | |
| MEK (1) | Toluene (4) | // | // | Clear solution |
| Acetonitrile | MTBE (15) | // | // | Clear solution |
| (5) | | | | |
| THF (0.3) | Water (0.4) | Form P | Form P | // |
| Ethyl | Heptane (8) | Form C | Form P | // |
| acetate (2) | | | | |
| DCM (7) | Heptane (16) | Form A | Form P | // |
| Isopropyl | MTBE (16) | // | // | Clear solution |
| acetate (3.5) | | | | |
| DMSO | Water (0.2) | Form C + | Form P | // |
| (0.3) | | Form G | | |
| 2-MeTHF | MTBE (2.4) | // | Form P | Clear solution |
| (0.6) | | | | After stirring at 25° C. for about 8 |
| | | | | days, clear solution became a |
| | | | | suspension. |

// = No comments.

A 6 g scale crystallization using typical process in 2-MeTHF/n-heptane system was carried out, using the following procedure, to prepare material for drying stability study and verify the process using typical crude API material.
1. Dissolve crude material in 6v 2-MeTHF at 60° C. into a reactor (R1) (solution)
2. Adjust R1 to 45-50° C.
3. Add 1 wt % seed (Form P) into R1
4. Stir R1 at 45-50° C. for 4 h
5. Charge 12v n-heptane into R1 dropwise over 10 h at 50° C.
6. Stir R1 at 45-55° C. for 2 h
7. Adjust R1 to 0° C. over 5 h
8. Stir R1 at 0° C. for 18 h
9. Filter and rinse with 2v 2-MeTHF/n-heptane ½ v/v for twice, total 4v
10. Dry at 50° C. by vacuum for 16 h
11. Obtain the material

This procedure was performed with XRPD showing Form P both times. Purity was measured at 100%.

### Cooling Crystallization

1. Dissolve 3 g Form P in 6v 2-MeTHF at 60° C. into a reactor (R1) (solution)
2. Adjust R1 to 45° C.
3. Charge 1% seed of (PC15541-3-FP-P) into R1.
4. Stir R1 at 45° C. for 1 h
5. Charge 12v Heptane into R1 dropwise over 2 h at 45° C.
6. Stir R1 at 45° C. for 1 h
7. Adjust R1 to 0° C. over 1 h (IPC1: take solution to test IPLC)
8. Stir R1 at 0° C. for 16 h (IPC2: take solution to test HPLC)
9. Filter and rinse wet cake with 2v*2 (2-MeTHF: Heptane=1:2)
10. Dry at 50° C. by vacuum for 24 h

### Wet Cake Chemical Stability

Wet cake chemical stability of Form P at 40° C., 50° C., 60° C. was evaluated. The details and results are shown in Table 16.

TABLE 16
Wet cake chemical stability

| | | Purity after holding for different time | | |
|---|---|---|---|---|
| Hold condition | Temperature | 0 h | 22 h | 4 d |
| Hold the wet | 40° C. | Purity: 100% | 100% | 100% |
| cake from | 50° C. | | 100% | 100% |
| crystallization | 60° C. | | 100% | 100% |
| into a sealed | | | | |
| bottle under | | | | |
| different | | | | |
| temperatures | | | | |

Results showed the wet cake of Form P is stable under 60° C.

### Dry Cake Chemical Stability

The cake of Form P was dried under vacuum at different temperatures to evaluate the chemical stability of dry cake. The results are listed in Table 17.

TABLE 17
Dry cake chemical stability

| | | Purity after holding for different time | | |
|---|---|---|---|---|
| Solid state | Temperature | 0 h | 22 h | 4 d |
| Dry the wet | 40° C. | Purity: 100% | 100% | 100% |
| cake after | 50° C. | | 100% | 100% |
| crystallization | 60° C. | | 100% | 100% |
| under vacuum at | | | | |
| different | | | | |
| temperatures | | | | |

Results showed the wet cake of Form P is stable under 60° C.

### Hygroscopicity

Hygroscopicity of Form P was evaluated by dynamic vapor sorption (DVS) test at 25° C. Form P is non-hygroscopic at 80% RH; about 0.1% water uptake at 80% RH. Form P is slightly hygroscopic from 80% RH-95% RH: About 1.6% water uptake from 80% RH to 95% RH. After the DVS test, the obtained sample was still in Form P. The results of these experiments are summarized in Table 18.

TABLE 18
Water sorption and desorption experiments of Form P

| | 40-0-95-0-40% RH, dm/dt 0.002, minimum equilibration time 60 min/step, | | | |
|---|---|---|---|---|
| Method | maximum equilibration time 360 min/per step, 25° C. | | | |
| Relative | 1^{st} desorp. | 1^{st} sorp. | 2^{nd} desorp. | 2^{nd} sorp. |
| humidity at | Weight % | Weight % | Weight % | Weight % |
| 25° C. | change | change | change | change |
| 0% | 0.1 | 0.1 | 0.0 | 0.0 |
| 10% | 0.1 | 0.1 | 0.0 | 0.0 |
| 20% | 0.1 | 0.1 | 0.0 | 0.0 |
| 30% | 0.2 | 0.1 | 0.1 | 0.0 |
| 40% | 0.2 | 0.1 | 0.1 | 0.0 |
| 50% | // | 0.1 | 0.1 | // |
| 60% | // | 0.1 | 0.2 | // |
| 70% | // | 0.1 | 0.3 | // |
| 80% | // | 0.2 | 0.4 | // |
| 90% | // | 0.8 | 0.9 | // |
| 95% | // | 1.8 | 1.8 | // |

// = No comments.
water uptake <0.2% = non-hygroscopic
water uptake ≥0.2% but <2% = slightly hygroscopic
Water uptake = water sorption in a specific RH (80% to 95%) - water sorption in 40% RH
The criteria are modified from the European Pharmacopeia criteria about hygroscopicity.

### Mechanical Properties

Dry grinding simulation experiments were conducted to evaluate viability of Form P over Form C.

About 10 mg of Form P was ground manually with a mortar and a pestle for 1, 3 and 5 minutes. Potential form change, and degree of crystallinity were evaluated by XRPD as shown in Table 19.

TABLE 19
Dry grinding simulation experiments of Form P

| Grinding time | XRPD | Comments |
|---|---|---|
| 1 min | Form P | No obvious change in crystallinity |
| 3 min | Form P | No obvious change in crystallinity |
| 5 min | Form P | No obvious change in crystallinity |

About 10 mg of Form C was ground manually with a mortar and a pestle for 5 minutes. Potential form change, and degree of crystallinity were evaluated by XRPD as shown in Table 20.

TABLE 20
Dry grinding simulation experiments of Form C

| Grinding time | XRPD | Comments |
|---|---|---|
| 5 min | Form C | Crystallinity decreased slightly |

Wet granulation simulation experiments were conducted to evaluate viability of Form P over Form C.

Water or ethanol was added drop wise to about 10 mg of Form P until the sample is wetted sufficiently. Wet sample was ground gently with in a mortar and a pestle. Post granulation sample was dried under ambient condition for 10 min. Potential form change, and degree of crystallinity were evaluated by XRPD as shown in Table 21.

TABLE 21
Wet granulation simulation experiments of Form P

| Granulation solvents | XRPD | Comments |
|---|---|---|
| Water | Form P | No obvious change in crystallinity |
| Ethanol | Form P | No obvious change in crystallinity |

Water or ethanol was added drop wise to about 10 mg of Form C until the sample is wetted sufficiently. Wet sample was ground gently with in a mortar and a pestle. Post granulation sample was dried under ambient condition for 5 min. Potential form change and degree of crystallinity were evaluated by XRPD as shown in Table 22.

TABLE 221
Wet granulation simulation experiments of Form C

| Granulation solvents | XRPD | Comments |
|---|---|---|
| Water | Form C | Crystallinity decreased slightly |
| Ethanol | Form C | Crystallinity decreased |

### Single Crystal Cultivation of Form P

About 200 mg of Form C (chemical purity 100%) was weight into 1 mL of EtOH. The obtained suspension was filtered through a 0.45p m syringe nylon membrane filter. Then the clear solution was slowly evaporated at 70° C. After for about 4 days, single crystals of Form P were obtained.

### Single Crystal Analysis of Form P

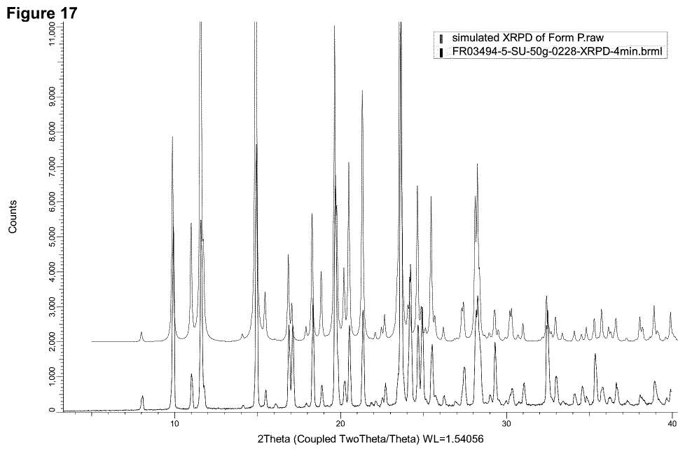

Form P was used for crystal structure determination. The single crystal structure of Form P was determined at 298(2) K. Form P is crystallized in monoclinic system, P2₁/c space group with Rᵢₙₜ=4.3% and the final R₁=[I>2σ(I)]=4.7% at 298(2) K. The Ortep image of the Form P was shown in FIG. 16. Simulated XRPD of Form P is in accordance with the experimental Form P data FIG. 17.

### Evaluation of Form

Form P is the optimal polymorph identified. Its stability, solubility, hygroscopicity and feasibility of formulation process were evaluated as described above.

Various modifications of the disclosure, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A crystalline form of 3-(5-(2-hydroxy-2-methylpropoxy)-6-methylpyrazin-2-yl)-1H-indole-7-carbonitrile, wherein the crystalline form is characterized by an XRPD diffractogram having peaks expressed in degrees-2-theta at angles (±0.2 degrees) of 23.7, 11.1, and 14.9.

2. The crystalline form of claim 1, wherein the crystalline form is characterized by an XRPD diffractogram having peaks expressed in degrees-2-theta at angles (±0.2 degrees) of 23.7, 11.1, 14.9, and 9.9.

3. The crystalline form of claim 1, wherein the crystalline form is characterized by an XRPD diffractogram having peaks expressed in degrees-2-theta at angles (±0.2 degrees) of 23.7, 11.1, 14.9, 9.9, and 19.7.

4. The crystalline form of claim 1, wherein the crystalline form is characterized by an XRPD diffractogram having peaks expressed in degrees-2-theta at angles (±0.2 degrees) of 23.7, 11.1, 14.9, 9.9, 19.7, 29.3, and 24.7.

5. The crystalline form of claim 1, wherein the crystalline form is characterized by an XRPD diffractogram having peaks expressed in degrees-2-theta at angles (±0.2 degrees) of 23.7, 11.1, 14.9, 9.9, 19.7, 29.3, 24.7, 21.4, and 17.1.

6. The crystalline form of claim 1, wherein the crystalline form is characterized by an XRPD diffractogram having peaks expressed in degrees-2-theta at angles (±0.2 degrees) of 23.7, 11.1, 14.9, 9.9, 19.7, 29.3, 24.7, 21.4, 17.1, 18.4, and 20.6.

7-8. (canceled)

9. The crystalline form of claim 1 having a DSC thermogram characterized by an endotherm with an onset temperature range 168.5-171.5° C.

10. The crystalline form of claim 1 having a DSC thermogram characterized by an endotherm with an onset temperature range of 179.5-180.5° C.

11. (canceled)

12. A pharmaceutical composition comprising the crystalline form of claim 1 and a pharmaceutically acceptable carrier.

13. A method of treating a neurodegenerative disorder in a subject in need thereof comprising administering to the subject a therapeutically effective amount of the crystalline form of claim 1.

14. The method of claim 13, wherein the neurodegenerative disorder is an x-linked recessive disorder.

15. The method of claim 13, wherein the neurodegenerative disorder is spinal bulbar muscular atrophy (SBMA).

16. A method of modulating androgen receptor (AR) activity in a subject in need thereof comprising administering to the subject a therapeutically effective amount of a compound of claim 1.

17. The method of claim 16, wherein the androgen receptor (AR) undergoes allosteric modulation.

18. The method of claim 16, wherein modulating androgen receptor (AR) activity treats spinal bulbar muscular atrophy (SBMA) in the subject.

19. A method of treating cancer in a subject in need thereof comprising administering to the subject a therapeutically effective amount of a compound of claim 1.

20. The method of claim 19, wherein the cancer is prostate cancer.

21. A process for preparing the crystalline form of claim 1 comprising:
a) combining 3-(5-(2-hydroxy-2-methylpropoxy)-6-methylpyrazin-2-yl)-1H-indole-7-carbonitrile with a polar, protic solvent to form a suspension;
b) stirring the suspension at between 35° C. and 40° C.;
c) collecting crystalline solids of 3-(5-(2-hydroxy-2-methylpropoxy)-6-methylpyrazin-2-yl)-1H-indole-7-carbonitrile by centrifugation.

22. (canceled)

23. A process for preparing the crystalline form of claim 1 comprising:
a) combining 3-(5-(2-hydroxy-2-methylpropoxy)-6-methylpyrazin-2-yl)-1H-indole-7-carbonitrile with a polar, aprotic solvent to form a suspension;
b) stirring the suspension at between 45° C. and 55° C.;
c) adding to the solution seeds of crystalline 3-(5-(2-hydroxy-2-methylpropoxy)-6-methylpyrazin-2-yl)-1H-indole-7-carbonitrile;
d) re-equilibrating the solids in the polar, aprotic solvent under a temperature cycle between about 5° C. to about 50° C.; and
e) collecting the crystalline form of 3-(5-(2-hydroxy-2-methylpropoxy)-6-methylpyrazin-2-yl)-1H-indole-7-carbonitrile.

24. A process for preparing the crystalline form of claim 1 comprising dissolving 3-(5-(2-hydroxy-2-methylpropoxy)-6-methylpyrazin-2-yl)-1H-indole-7-carbonitrile in a solvent system to form a suspension, heating the suspension to between 45° C. and 55° C., passing the suspension through a filter to isolate a solution, cooling the solution to form a slurry, and filtering the slurry to isolate the crystalline form of 3-(5-(2-hydroxy-2-methylpropoxy)-6-methylpyrazin-2-yl)-1H-indole-7-carbonitrile.

25-41. (canceled)
